(19) United States
(12) Patent Application Publication
(10) Pub. No.: US 2025/0009229 A1
(43) Pub. Date: Jan. 9, 2025

(54) METHODS AND PROCESSES FOR LOCALIZATION OF TUMORS THROUGH VIBRATIONAL TECHNIQUES

(71) Applicant: Emanuele Raggi, Providence, RI (US)

(72) Inventor: Emanuele Raggi, Providence, RI (US)

(21) Appl. No.: 18/764,302

(22) Filed: Jul. 4, 2024

Related U.S. Application Data
(60) Provisional application No. 63/511,889, filed on Jul. 4, 2023.

(51) Int. Cl. A61B 5/00

(52) U.S. Cl. CPC A61B 5/0051; A61B 5/4312; A61B 5/4842

(57) ABSTRACT

Methods and processes for localization of tumors through vibrational techniques are disclosed to identify suspect malignancies within a body part or an organ. Directing two pulsed wave photoacoustic excitation sources working simultaneously and in synchronization into a desired area of the body distributing acoustic energy into the tissue at the speed of sound and detecting the surface wave on the tissue may reveal specific vibrational patterns that are specific for health and cancerous tissues. These methods rely on the analysis of vibrational patterns using image post-processing techniques, numerical modeling or a digital library. The use of non-contact techniques can be used for breast cancer leading to an improved prognosis.

### CROSS-REFERENCE TO RELATED APPLICATION

This application claims the benefit of the U.S. Provisional Application No. 63/511,889 entitled, “METHODS AND PROCESSES FOR LOCALIZATION OF TUMORS THROUGH VIBRATIONAL TECHNIQUES” filed on Jul. 4, 2023, which is incorporated herein by reference in its entirety.

### TECHNICAL FIELD

Embodiments relate generally to methods and processes to localize tumors completely contactless exploiting the vibrational pattern analysis of optically detected surface vibrational wave modes.

### STATEMENT REGARDING FEDERALLY SPONSORED RESEARCH

N/A

### BACKGROUND OF THE INVENTION

Up to 30% of breast cancer diagnoses are missed in the early stages (false-negative). False-negative results lead to delays in treatment and a false sense of security for affected women. Yearly breast cancer diagnoses are massive, making this an extremely serious problem. In the USA close to 288,000 women were diagnosed with breast cancer in 2022 and approximately 43,000 died due to a late diagnosis. Additionally dense breasts of younger women precludes the detection of early-stage tumors by mammography. Triple negative breast cancer (TNBC) is the most aggressive and 12% of women are yearly affected. A dense breast plays a significant role in missing a TNBC diagnosis at the early stage, decreasing the overall survival rate for the affected women. Limitations of the current mechanical transducers used in conventional sonography are mainly due to resonant devices with rather limited operating-frequency ranges and little modulation agility. Thus, there is an unmet clinical need for accurate, painless, cheap early detection methodology in the current ultrasound market.

This patent will disclose a non-contact optical surface vibrational technique analysis that is able to differentiate healthy tissue from cancerous tissues. The innovative idea is the absence of usage of gel, water or electrodes while performing an ultrasound session, saving the patient's pain due to the sonographers application of pressure on the probe, causing discomfort in the majority of cases, and eliminating the sonographer's chronic pain. A combination of two lasers working in synchronization with an external optical interferometer are able to detect skin surface waves. Given the vibrational response of the tissue to the external laser excitation, it is possible to differentiate a healthy to a cancerous tissue by understanding the surface vibrational pattern. Intensity of surface vibration results in the creation of a stiffness map, which forms the final image.

One obvious advantage of laser-based sonography is the higher comfort level to the patient due to its contact-less nature. Other advantages in laser-based sonography are due to the easy control of laser light with electronic means and laser pulses can be generated at almost any temporal pattern, thus allowing single pulses, continuous-wave operation, deep amplitude and frequency modulation. Finally, laser-based methods allow for much more agile scanning across the patient than any mechanical transducer would be capable of. In 2021 we conducted several field interviews with doctors and patients to assess the market concluding that 90% of interviewed patients would prefer not to be touched during an ultrasound session and 77% would go contactless due to the “less invasive” approach. 83% of patients would prefer more automation inside a hospital as they are aware of the danger of missed diagnoses.

The present invention relates to systems and methods for generating 2D and 3D images without applying any contact to the surface of the patient, only detecting and processing how the vibration on the skin of the patients is received and analyzed can provide a terrific improvements in patient's experience a healthier way of localizing certain diseases given the non-contact process used.

An ultrasound wave occurs at frequencies that are above the bandwidth of human audio capabilities. In terms of imaging, the frequencies cover a wide variety of applications, including, but not limited to, underwater sonar systems, industrial non-destructive testing, near-surface damage assessment, medical diagnostics, and evaluation of acoustic micro-structures.

Regular ultrasonic imaging techniques have long-known benefits. Technological advancements in hand-held probes have been proven to provide better images for soft tissue structures such as the eyes, abdomen, brain, neck, and feet. Conventional techniques for obtaining ultrasound images typically require applying a mechanical probe directly to the patient's body, focusing on the specific area of interest for a particular period of time. The amount of time spent probing is determined by the sonographer's level of experience and by the complexity of the anomaly being investigated.

During an ultrasound, the probe emits an acoustic wave, causing a thermal dilation of tissue that consequently deforms and elasticizes the tissue, generating a returning wave. These waves ricochet back to the probe, and the time difference between the original emission from the probe and the returning acoustic wave compose the sonogram, which is typically displayed on a monitoring system.

Unlike CT scans, MRIs, or x-rays, ultrasound imaging technologies are relatively low-cost and do not involve the injection of radiation or contrast. They are also portable. Ultrasound technicians and doctors must constantly balance the quality of scans with the hospitals' needs for efficient, high volume scanning to increase healthcare reimbursements.

The use of hand held probes, however, poses problems that current technology is not yet able to overcome. The evidence is clear that overuse injuries brought on by repetitive muscle stresses associated with the performance of ultrasound exams can lead to muscular damage and, in some cases, career-ending injuries. In addition, the hand-held probe may exacerbate existing pain for patients when technicians apply pressure on the point or area under investigation. This is especially prominent during a prolonged pressure application to a painful area.

Patients may also move during the procedure due to discomfort or pain, and this can interfere with the quality of the scan, corrupt the ultrasonic reading, or elongate the duration of the exam. If patients are in extreme pain or cannot be still during a scan, the hospital may have to reschedule a session, since efficiency and patient turnover must remain high in the current profit structure.

A patient's body type and size also impact the quality of an ultrasound. With overweight and obese patients, the ultrasound wave has to pass through more fat tissue before reaching the main site, sometimes causing an unclear reading. In addition, internal gas can also interfere with the accuracy of a reading, requiring a longer session. In these cases, the sonographer must apply a great deal of pressure to the site.

In order to produce an accurate reading using current ultrasound technology, the system must always have a clear understanding of where the probe is in space. Spatial resolution is often a major obstacle with handheld devices. Accurate readings require the system to have a continuous record of the angular velocity, linear velocity, roll, pitch, and yaw. While current technology offers spherical probe transducers, which concentrate the beam, there is still too much room for human error. For example, if the technician's hand trembles or if there is inconsistency in the amount of pressure they apply, this can compromise the integrity of the reading. In addition, if the sonographer did not apply enough gel to create the ideal electrodynamic balance, the returning wave could be flawed and lead to an inaccurate scan.

In conventional sonography, a medical practitioner applies a sono-mechanical transducer to the skin or interior surfaces of a patient as close as possible to the organ of interest. The transducer both launches sound waves, and records echoes, which are caused by variations of sound-propagating properties (impedance) in the tissue. Because the sound impedance is specific for each tissue type, the echoes are correlated to physiological features. The time between launching of sound waves and reception of echoes gives the depth below the point where the transducer has been applied.

Recently, contactless sonography methods have been tested where rapid pulsed heating of the skin due to illumination with a pulsed laser generates sound waves and laser-interferometric methods measure the vibration of the skin due to returning echo. One obvious advantage of laser-based sonography is the higher comfort level to the patient due to its contact-less nature. There are, however, other less immediately obvious advantages in laser-based sonography due to the easy control of laser light with electronic means: Whereas the mechanical transducers used in conventional sonography are resonant devices with rather limited operating-frequency ranges and little modulation agility, laser pulses can be generated at almost any temporal pattern, thus allowing single pulses, continuous-wave operation, deep amplitude and frequency modulation, and so on. This capability allows the implementation of sophisticated sensing techniques that are based on recognizing and categorizing vibrational patterns.

Finally, laser-based methods and optical vibrational-based methods allow for much more agile scanning-detection across the patient than any mechanical transducer would be capable of. To our knowledge, current work is not yet exploiting the advantages of the more sophisticated sensing schemes.

Research and experimentation into pulse or continuous lasers to be used as contactless ultrasonic devices is in the initial stages. Some studies have proven that contactless ultrasound techniques can be a useful tool for both humans and animals. Eye-safe techniques are already being used to treat optical problems such as myopia and are considered safe at a particular exposure. Wireless portable ultrasound probes are already in use and present a great advantage in terms of portability and field operations. They do not, however, eliminate the need to physically touch the patient nor do they limit sonographers' stress injuries. This demonstrates a need for developing novel techniques and processes in this field that allow for acoustic waves to enter soft tissue, skin, and organs without the need for physical contact with the patient and based on the vibration that is received on the surface of the skin, precisely categorize that vibration.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later. In order to overcome the aforementioned limitations in current detection technology, the present invention utilizes non-contact optical vibration-based techniques on the skin tissue to reveal specific vibrational patterns that are specific for health and cancerous tissues. These methods rely on the combined analysis of mechanical vibrational patterns, surface charge distribution, stress-strain analysis and pressure field distribution using image post-processing techniques, numerical modeling or a digital library.

The use of non-contact techniques can be used for breast cancer leading to an improved prognosis.

The application of laser ultrasonics for clinical diagnostic assessment of the internal structure of tissues carries significant advantages over current models. A pulse laser, or Q-switched laser, may be used for internal localization and detection of features or soft tissues by emitting a photoacoustic ultrasonic wave toward the patient. The return wave is represented by a coherent summation of waves that present themselves on the patient's skin and are analyzed with an optical tool such as an interferometer. When the returning wave ricochets back to the surface it presents itself as a vibration at that specific point. That vibration corresponds to the maximum vibration detected at that point using the optical interferometer. The vibrational point is then captured with the interferometer and given the proper xyz coordinate, in addition to that a mechanical analysis of the vibration can reveal the stress-strain status of the tissue calculating the Elastic Modulus at that specific point, a pressure field association can also be performed at that specific point, calculation of charge distribution can also be performed. A higher charge distribution is typically associated with a more damaged tissue and finally the sound impedance is specific for each tissue type. All these parameters are correlated to physiological features that overall can provide information on the status of the tissue. A total of four different maps can be created such as: (1) vibrational map, (2) stiffness map, (3) pressure map and finally a (4) charge map. Those four maps can be overlapped to obtain a complete tissue categorization and understand if the doctor is observing a cancerous or healthy tissue. VSPC (vibrational, stiffness, pressure, charge) is the acronym that can be used.

The VSPC map displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This VSPC map would show how mechanical properties of tissue change across different locations of the scanned area.

One embodiment of the present invention provides a method for generating 2D/3D vibrational images of a patient based on tissue vibrational wave modes. Unlike conventional ultrasonic efforts to establish the internal structure of soft tissues or organs and understand if the observer is looking at a healthy or a cancerous tissue. Laser generation techniques, being contactless, do not require any specific preparation of the skin. The method includes generating ultrasonic acoustic waves using pulsed wave lasers. The beam of the excitation source is focused on a specific area of interest of the body and emits ultrasonic waves.

Due to backscattering, returning acoustic waves are detected via interferometric systems. Detected waveforms on the skin of the patient are subsequently analyzed and processed to evaluate whether the doctor is observing a cancerous or a healthy tissue.

Detected waveforms on the skin of the patient are subsequently analyzed by a processor which provides information to evaluate part of the body or an internal organ.

Another embodiment of the present invention provides a method for generating 2D/3D vibrational maps and stiffness maps a patient to establish the internal structure of soft tissues or organs and understand if the observer is looking at a healthy or a cancerous tissue. The method includes generating ultrasonic acoustic waves using pulsed wave lasers. The beam of the excitation source is focused on a specific area of interest of the body and emits ultrasonic waves.

Another embodiment of the present invention provides a method for generating 2D/3D vibrational maps, stiffness maps and pressure field maps of a patient to establish the internal structure of soft tissues or organs and understand if the observer is looking at a healthy or a cancerous tissue. The method includes generating ultrasonic acoustic waves using pulsed wave lasers. The beam of the excitation source is focused on a specific area of interest of the body and emits ultrasonic waves.

Another embodiment of the present invention provides a method for generating 2D/3D vibrational maps, stiffness maps, pressure field maps and charge distribution maps of a patient to establish the internal structure of soft tissues or organs and understand if the observer is looking at a healthy or a cancerous tissue.

Another embodiment of the present invention provides a method for generating 2D/3D vibrational maps, stiffness maps, pressure field maps, charge distribution maps or any combination of those maps of a patient to establish the internal structure of soft tissues or organs and understand if the observer is looking at a healthy or a cancerous tissue.

Still another embodiment of the present invention provides a method for generating 2D/3D ultrasound images of a patient by displaying an ultrasound view of an anatomic structure derived from laser-based sources. The system works by applying the concept of superposition to overlap layers of images taken from the various cameras and lasers, aligning with the position and orientation of the exteroceptive sensors in order to accurately reconstruct the images. This embodiment can be divided into the following steps: a) since a patient may move during the ultrasound session, the sonographer or the doctor can highlight specific points on the skin of the patient with a highlighter allowing the cameras to feature-detect those points and have a specific search area that keeps the reference frame stable; b) laser-based ultrasonic sources start emitting pulses allowing for surface wave detection; c) the interferometer detects those maximum vibrational waves within the area and assign xyz location to every point; d) all those points are combined together to create a VSPC map at that specific depth or a combination of those maps; e) laser(s) is/are regulated to have a higher or lower penetration rate so it is possible to create another layer (another VSPC map or a combination of those maps) at a different depth but within the same area mentioned at point a); f) repeat process e) for several depth so that the sonographer can form different layers at different depths of the same search area; g) interpolate the different layers in order to form a 3D image completely contactless and obtain at the same time information related to stress of the tissue, mechanical vibration, Yung's modulus, charge distribution, impedance and pressure field.

Additionally, image reconstruction may be derived from non-parametric filtering techniques, included but not limited to, histogram filters, particle filters and gaussian probability hypothesis density filters.

Additional features and advantages of the various aspects of the present invention become apparent from the following description of its preferred embodiments. The description provided should be taken in consideration with the accompanying drawings. The aforementioned embodiments do not represent the full scope of the invention. The references following the description of the preferred embodiments are made thereof to the claims and herein for interpreting the scope of the present invention.

Other objects, features and advantages of the invention shall become apparent as the description thereof proceeds when considered in connection with the accompanying illustrative drawings.

### BRIEF DESCRIPTION OF THE DRAWINGS

The novel features that are characteristic of the present invention are set forth in the appended claims. However, the invention's preferred embodiments, together with further objects and attendant advantages, will be best understood by reference to the following detailed description taken in connection with the accompanying Figures:

FIG. 1 is an image showing the final result of obtaining an image using non-contact laser techniques. In the image every single point represents the point where the laser was shot. Different colors are a representation of a different vibrational intensity.

FIG. 2 is an image showing the final result of obtaining an image using non-contact laser techniques. Points following in the same vibrational intensity are in the same square. Points with different intensity are enclosed in a different square.

FIG. 3 is an image showing the final result of obtaining an image using non-contact laser techniques. Points following in the same vibrational intensity are in the same square. Points with different intensity are enclosed in a different square. The square that encloses points with the same vibrational intensity takes the same color of the points for interpolation techniques. This way the image is smoother. Horizontal and vertical red rectangles are representing vasculature around the tumor.

FIG. 4 is an image showing the square that encloses points with the same vibrational intensity and that takes the same color of the points. This image smoothes the squares and discretizes the image into squares with different colors. Different colors are a representation of different vibrational patterns.

FIG. 5 is an image showing the central close necrotic core of the tumor. This is a discretization of the tumor close core and their vibrational response is the same across the tumor interface.

FIG. 6 is an image showing the central far necrotic core of the tumor. This is a discretization of the tumor far core and their vibrational response is the same across the tumor interface. Colors are different from FIG. 5 because at the borders there is a different pressure field, therefore a different vibrational response.

FIG. 7 is an image showing the glycolytic area surrounding the tumor core. Colors are different from FIG. 6 and FIG. 7 because in this area there is a different pressure therefore a different vibrational response. This is the case where the interstitial pressure plays a significant role in terms of response to therapy.

FIG. 8 is an image showing the healthy tissue surrounding the glycolytic area. Across the blue area the vibrational response is the same.

FIG. 9 is an image showing the entire composition of the different areas characterized by different vibrational response and color with the addition of vasculature to show the blood flow direction.

FIG. 10 is an image showing an enlargement of FIG. 3. The red box representing the bigger image represents an area with different vibrational points and, consequently, different vibrational averaged square color. The enlargement of the tumor host interface in green is representing the probability that a specific square has the same specific color of the neighboring squares. If the probability is high enough the same color is assigned to that vibrational point.

FIG. 11 is an image showing an enlargement of FIG. 10 for clarity reasons.

FIG. 12 is an image showing how the vibrational algorithm assignment works. Add scanning points (and create squares). Is the vibration comparable to the 3 squares that are touching square 3 depicted in purple? If the answer is yes, then the algorithm will update previous probability and assign the same color intensity because the vibration is comparable to the other neighboring squares; then the algorithm proceeds to calculate pressure field, surface charge field, impedance and stress-strain and finally update the kernel. If the answer is no, then the algorithm will update previous probability and assign a different color intensity because the vibration is not comparable to the other neighboring squares; then the algorithm proceeds to calculate pressure field, surface charge field, impedance and stress-strain and finally update the kernel.

FIG. 13 is an image showing how the vibrational algorithm assignment works. This image is a simple expansion to the explanation of FIG. 12.

FIG. 14 is an image showing how the vibrational algorithm assignment works. This image is a simple expansion to the explanation of FIG. 12. With the difference that at this point the algorithm will foresee what the next color assignment will be based on the previous outcomes.

FIG. 15 is an image showing how the vibrational algorithm assignment works in case the vibrational intensity of a point is the same as the neighboring points (or squares). The color assignment is the same as the others.

FIG. 16 is an image showing how the vibrational algorithm assignment works in case the vibrational intensity of a point is not the same as the neighboring points (or squares). The color assignment is not the same as the others.

FIG. 17 is an image showing an enlargement of the area with the same vibrational intensity. The numbers within the square are representing how many squares are bordering that specific square. The green dots are a representation of the scanned point. The green square with the four green points at the angle is a representation of a smooth average surface that has the same vibrational intensity as the points.

FIG. 18 is an image showing how the color probability is assigned. The probability is assigned based on three different values: (1) the border probability, (2) the area probability and (3) the point probability. These three probabilities combined together provide the final probability square and the related vibrational assignment.

FIG. 19 is an image showing how a bell curve can be composed of just points.

FIG. 20 is an image showing how a bell curve can be composed of just grid lines.

FIG. 21 is an image showing how a bell curve can be composed of just area squares.

FIG. 22 is an image showing how a bell curve can be better characterized combining FIG. 19, FIG. 20 and FIG. 21

FIG. 23 is an image showing a specific area such as the one in FIG. 10 in the bigger red box. This image shows how after the scanning points are created, averaged into squares and assigned a specific color. In this image we wanted to show how the points have a specific number. The sequential number represents the scanning direction and the pulse coordinates of the laser. There is the point probability, line probability and area probability, which combining those probabilities will attribute a specific vibrational intensity, corresponding to a specific color which is also dependent on the neighboring squares.

FIG. 24 is an image showing how the overall vibrational assignment happens and the scanning direction of the laser. Down right of FIG. 24 is the scanning direction. This is the representation of a specific layer at a specific depth within a specific area of interests.

FIG. 25 is an image showing how the overall image is obtained. Which in this case is a composition of VSPC (vibrational, stiffness, pressure, charge). Or it could also be a combination of just some of those maps.

FIG. 26 is an image showing a table related to human value at rest for blood flow.

FIG. 27 is an image showing a table related to human value at rest for specific heat capacity.

FIG. 28 is an image showing a table related to human value at rest for density.

FIG. 29A is an image showing a table related to human value at rest for thermal conductivity.

FIG. 29B is an image showing a table related to human value at rest for thermal conductivity.

FIG. 30A is an image showing a table related to human value at rest for water content.

FIG. 30B is an image showing a table related to human value at rest for water content.

FIG. 31 is an image of a flowchart showing how the process of forming an image is structured

FIG. 32 is an image of a flowchart showing how the process of forming a vibrational and pressure image field is structured

FIG. 33 is an image of a flowchart showing how the process of forming a vibrational, pressure and surface charge distribution image field is structured

FIG. 34 is an image of a flowchart showing how the process of forming a vibrational, pressure, surface charge and stress-strain distribution image field is structured

FIG. 35 is an image of a flowchart showing how the process of forming an image is structured with comparison with routine images

FIG. 36 is an image of a flowchart showing how the process of forming a vibrational and pressure image field is structured with comparison with routine images

FIG. 37 is an image of a flowchart showing how the process of forming a vibrational, pressure and surface charge distribution image field is structured with comparison with routine images

FIG. 38 is an image of a flowchart showing how the process of forming a vibrational, pressure, surface charge and stress-strain distribution image field is structured with comparison with routine images

FIG. 39 is a flowchart showing a regular methodology for tumor localization

FIG. 40 is a flowchart showing methodology A for tumor localization

FIG. 41 is a flowchart showing methodology B for tumor localization

FIG. 42 is a flowchart showing methodology C for tumor localization

FIG. 43 is a flowchart showing methodology D for tumor localization

FIG. 44 is a flowchart showing methodology E for tumor localization

FIG. 45 is a flowchart showing methodology F for tumor localization

FIG. 46 is a flowchart showing methodology G for tumor localization

FIG. 47 is a flowchart showing methodology H for tumor localization FIG. 48 is am image of a phantom material mimicking a slice of the human breast

and the extraction of the vibrational map

FIG. 49 is an image showing both the vibrational and pressure field maps

FIG. 50 is an image showing both the vibrational, pressure field and surface charge map

FIG. 51 an image showing both the vibrational, pressure field, surface charge and stress-strain map. Altogether this forms the VSPC map

FIG. 52 is an image showing the combined VSPC image into a 3D image.

### DETAILED DESCRIPTION OF THE INVENTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

For convenience, the meaning of some terms and phrases used in the specification, examples, and appended claims, are listed below. Unless stated otherwise or implicit from context, these terms and phrases have the meanings below. These definitions are to aid in describing particular embodiments and are not intended to limit the claimed invention. Unless otherwise defined, all technical and scientific terms have the same meaning as commonly understood by one of ordinary skills in the art to which this invention belongs. For any apparent discrepancy between the meaning of a term in the art and a definition provided in this specification, the meaning provided in this specification shall prevail.

“Charge Map (CM)” has the mathematical art-defined meaning. The CM or Surface Charge Map (SCM), which terms would be used interchangeably over the length of this patent, displays magnitude as color in a two-dimensional matrix and assigns “+” or “−” signs to surfaces that may be positively or negatively charged due to an external stimulus of any type. This map would show how charge is distributed across different locations of the scanned area under investigation and particularly on the surface of every discretized square area. The variation in color may be by hue or intensity, giving obvious visual cues to the user about how the phenomenon varies over space. Also it would relate to the specific vibration. Vibration and surface charge are strictly connected here.

“Data Acquisition System (DAS)” has the electronic hardware art-defined meaning. A DAS for the context of this patent is intended to be an oscilloscope which is able to process data originating from the backscattering of ultrasonic waveforms on the surface of the tissue.

“Data Log (DL)” has the database art-defined meaning. A DL for the context of this patent is intended to be a storage system such as a database, where all information from external sensors are continuously stored in real-time. Such information is, for example, displacement, position, velocity and acceleration.

“External Visualizer (EV)” has the hardware art-defined meaning. The EV is intended as a regular computer monitor. A 3D visualizer is also a computer monitor where to show the 3D image. For the context of this patent external visualizer and 3D visualizer are intended as regular computer monitor and these terms will be used interchangeably.

“Filtering Techniques (FT)” has the mathematical robotic art-defined meaning. FT processes are algorithms aimed at finding patterns in historical and current data to extend them into future predictions, providing insights into what might happen next. Filtering techniques can be divided into parametric and non-parametric as explained below.

“Laser Stereometry (LS)” has the mathematical robotic art-defined meaning. A laser stereo image contains two views of a scene side by side. One laser view is intended for the left eye and the other laser view for the right eye.

“Non-Parametric Filter” has the mathematical robotic art-defined meaning. Non-parametric filters do not rely on any specific parameter settings and therefore tend to produce more accurate results. Non-parametric filters approximate posteriors by a finite number of values, each roughly corresponding to a region in state space. Examples of non-parametric filters are: histogram filter, particle filter, PHD filter, gaussian mixture PHD filter.

“OCTAVE” has the computer software art-defined meaning. Also known as GNU OCTAVE is a scientific programming language specifically used for scientific computing and used mainly both for numerical simulation and building graphical user interfaces. It is completely free and open source.

“Odometry” has the mathematical robotic art-defined meaning. Odometry is a common method of determining an object's motion from the way in which subsequent images overlap.

“Particle Filter (PF)” has the mathematical robotic art-defined meaning. The PF is a non-parametric solution to the Bayes Filter which uses a set of samples or “particles”. Each one of these particles can be seen as the possibility of an object being at that position at that time. PF is divided into three main steps. Prediction or state transition model, where the particle's position is predicted according to external sensors. Weighting or measurement model, where particles are weighted according to their likelihood with data provided by external sensors, and finally, resample, where particles with small weights are discarded.

“Pressure Map (PM)” has the mathematical art-defined meaning. The PM displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This map would show how pressure changes across different locations of the scanned area under investigation and particularly on the surface of every square discretized area. The variation in color may be by hue or intensity, giving obvious visual cues to the user about how the phenomenon varies over space. Also it would relate to the specific vibration. Vibration and pressure are strictly connected here.

“Processing System (PS)” has the hardware art-defined meaning. PS or Processor System is a computer such as a laptop or tower desktop able to process data in real-time or post-processing or pre-processing originated from a scanning session.

“Posterior” has the statistics art-defined meaning. A posterior, also known as posterior probability, in Bayesian statistics, is the revised or updated probability of an event occurring after taking into consideration new historical information in order to predict what might happen next. The posterior probability is calculated by updating the prior probability using Bayes' theorem. The posterior probability is the probability of event A occurring given that event B has occurred.

“Prior” has the statistics art-defined meaning. A Prior also known as prior probability, in Bayesian statistics, is the probability of an event occurring before new (posterior) data is collected.

“Stereo Images (SI)” has the mathematical robotic art-defined meaning. A stereo image contains two views of a scene side by side. One of the views is intended for the left eye and the other for the right eye. A stereo pair of images taken at each view's location is used to find distance. Then, the image from one camera is registered to the same camera's previous image, correcting rotation and scale differences. This registration is used to find the area's motion and speed between imaging positions.

“Stress-Strain Map (SM)” has the mathematical art-defined meaning. The SM displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This map would show how stress-strain is distributed across different locations of the scanned area under investigation and particularly on the surface of every square discretized area. From a stress-strain map it is possible to create a stiffness map. So stress-strain map and stiffness map are used interchangeably over the course of this patent. The variation in color may be by hue or intensity, giving obvious visual cues to the user about how the phenomenon varies over space. Also it would relate to the specific vibration. Vibration and stress-train are strictly connected here.

“Vibrational Map (VM)” has the mathematical art-defined meaning. The VM displays magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth. This map would show how vibration is distributed across different locations of the scanned area under investigation and particularly on the surface of every square discretized area. From a vibrational map it is possible to extrapolate several useful measurements such as pressure filed, surface charge and stress-strain. The variation in color may be by hue or intensity, giving obvious visual cues to the user about how the phenomenon varies over space. Also it would relate to the specific vibration.

“Vibrometric Sensor (VS)” has the robotic art-defined meaning. For the context of this patent a VS or interferometer have the same meaning. These instruments are able to optically detect surface vibrations. Therefore, these terms will be used interchangeably in this patent.

“Visual Inertial Odometry (VIO)” has the mathematical robotic art-defined meaning. VIO is a common method of determining an object's motion from the way in which subsequent images overlap.

“VSPC map” or “VSPC” has the mathematical imaging art-defined meaning. VSPC is the combination of vibrational, pressure field, surface charge and stress-strain map. Altogether this forms the VSPC map which could be shown as a 2D or 3D image. All the maps are composed by an image registration process and the color intensities are provided by a specific algorithm which will be further described in thi patent.

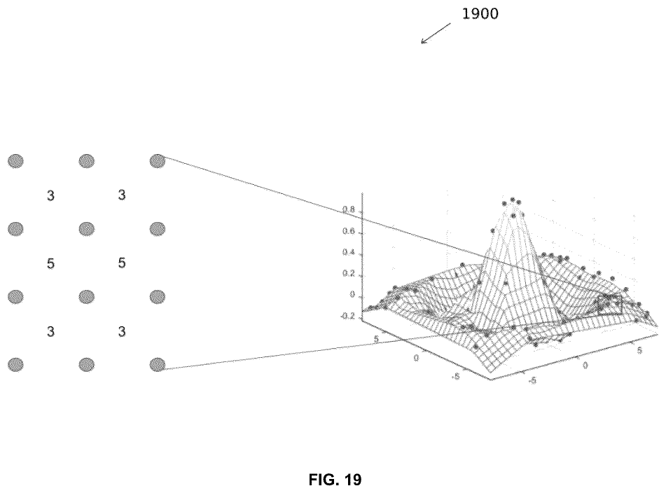

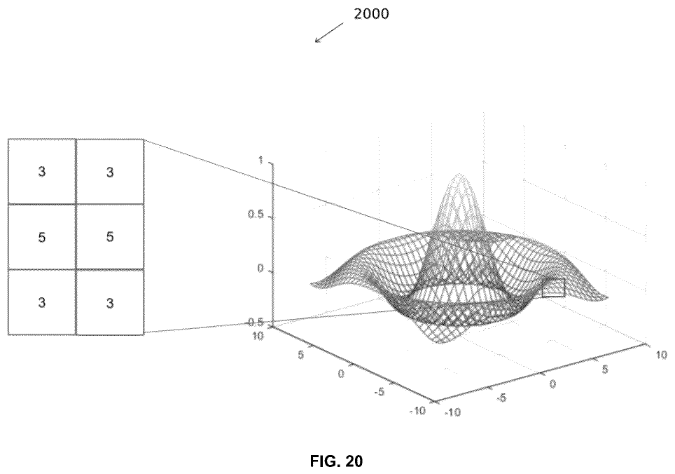

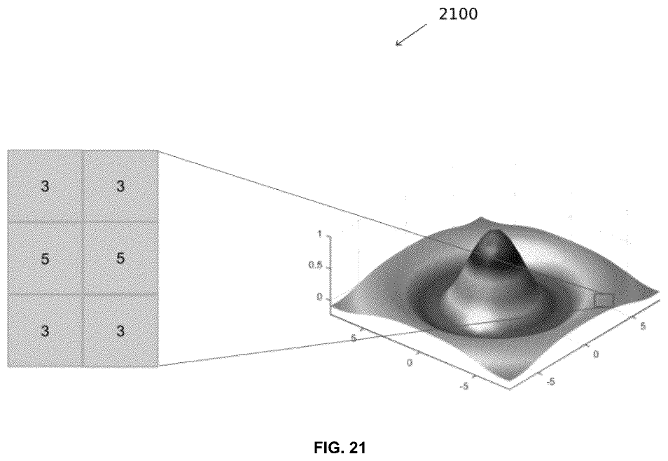

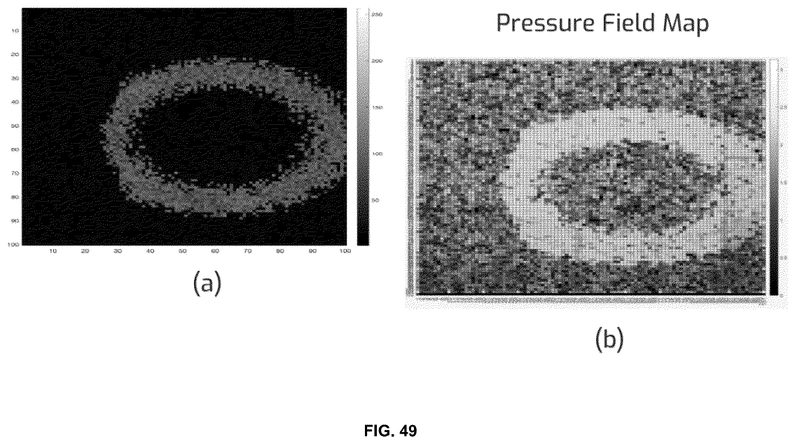

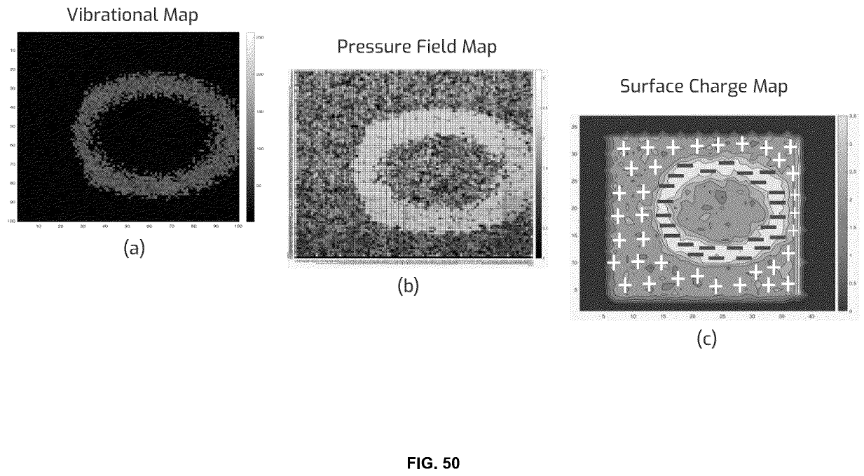

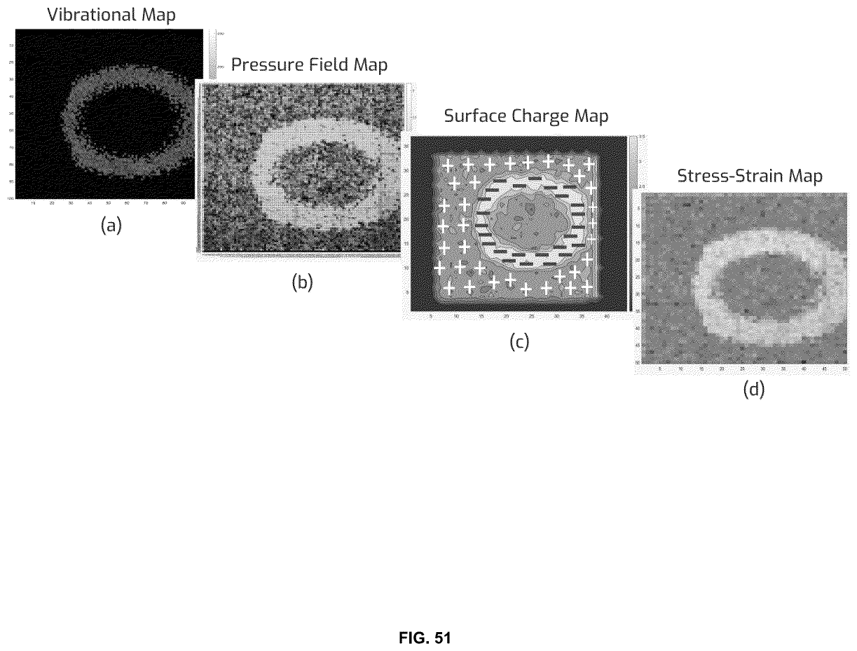

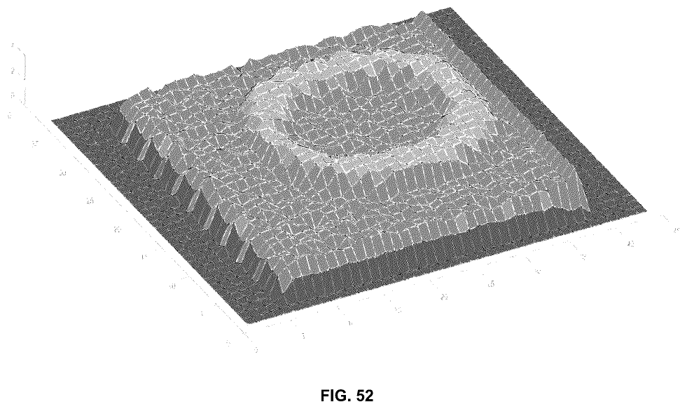

All drawings in FIG. 19, FIG. 20, FIG. 21, FIG. 48(c), FIG. 49, FIG. 50, FIG. 51 and FIG. 52 are made with the open source and freely available OCTAVE.

It would be appreciated by those skilled in the art that various changes and modifications can be made to the illustrated embodiments without departing from the spirit of the present invention. All such modifications and changes are intended to be within the scope of the present invention except as limited by the scope of the appended exemplary claims.

While there is shown and described herein certain specific structure embodying the invention, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

The main inventive concept has been described and many detailed drawings have been provided to clearly and precisely explain the concepts and how obtaining non-contact images from a laser excitation source analyzing a combination of surface vibration, pressure field, surface charge distribution and stress-strain (stiffness) maps and the idea behind the superposition algorithm. Those maps can all be superimposed to obtain precise and specific information about a specific part of the body or organ. Additionally, simply a combination of those considering those maps alone is also a source of great information. As mentioned in our previously filed Provisional Patent application every drawing will be described more in depth and in more details in this patient and the underlying mechanism to create images are also now described in greater detail below.

The present invention deals with providing vibrational, geometric, pressure, mechanical and electronic identifiers, specific markers and methodologies to identify the presence of tumors from a composition of various imaging techniques such as: vibrational images maps, stress-strain maps, pressure field maps, camera images and surface charge map of the body part. These images are obtained from external sensors such as: one black/white camera, one color camera, and a vibrometer used to detect surface vibration from an external stimulus such as a pulsed-laser (or a combination of synchronized pulsed lasers). The inventive concept behind this invention is the superposition of the various imaging techniques to identify in great detail a suspected malignancy. This patent specifically addresses finding breast cancer (or suspected breast cancer). However, given the nature of the invention, this patent can be used to uncover suspected malignancies in any part or tissue of the body, human or animal. This patent explains different methodologies for tumor detection and since the breast there is no presence of empty space or space between organs, fat or tissue but rather is a spaceless part of the body, there are no specific external or internal factors that may alter, distort or modify the resulting VSPC imaging map. The only piece of equipment needed (but typically already present in a hospital or healthcare clinic) is a sitting device such as a chair. Gravitational deformation of the breast due to sitting position does not represent a problem when proceeding with the imaging process and the construction of the VSPC imaging map. In this patent, breast refers to a body part that undergoes imaging, however a body part is referred to any body parts or organs both in the human and animal realm that have the capability to be clinically images. The result of the VSPC map can either be interpreted by the curing physician or be transmitted to an imaging center to be further evaluated if there is any need.

There are important steps that need to be described before arriving at the final VSPC map. The patient enters the imaging room and sits on a sitting device such as a chair. Hospital clothing would be provided for privacy reasons such as a gown with an opening in the front so that the breast is exposed and ready for imaging. Contrarily to other imaging modalities such as thermography, the patient does not need any acclimation periods to reach the steady state condition of blood flow irrorating the breast and the suspected malignancy. With the present invention, the patient may start the imaging process immediately after being seated. During the imaging process several images are taken using the external camera system described in order to create stereo-images and an external stimulus such as a pulsed Q-Switched laser scans the suspicious area. Images using the camera system are taken in a matter of seconds for a total of 10-20 images around the breast in order to have a geometrical digital stereo reconstruction of the breast. After that the external laser can start the scanning process which will take approximately 10-15 minutes to cover the area indicated by the doctor. Images taken with the camera can be further processed using software techniques or other numerical simulations to characterize the appearance of the breast and look for regions of interest. VSPC map obtained can also be further processed using software techniques or numerical simulation to characterize the malignancy such as: conspicuity, energy released by the tumor, size, location. The same philosophy can be applied to characterize the healthy surrounding tissue.

The angle and focus of the camera system can be different from the size and tissue type being screened and the same applied to the external laser system. However, what is important to note here is that orientation, roll, pitch and yaw of the camera system may be used to obtain other images for further studies. For the external laser system, its angle, focus or energy transmitted into the breast may also vary depending on the needs of obtaining additional images of neighboring parts, if needed.

After the screening process of the breast tissue is concluded and the VSPC imaging map, or a combination of those (e.g. vibrational map and pressure field map, or vibrational map and stress-strain map or vibrational map, stress-strain map and pressure filed map or vibrational map and surface charge map or vibrational map, stress-strain map and surface charge map) is created, the results are analyzed to see if a tumor is present or not. The regions of interest are therefore a combination of VSPC profiles that are indicative or abnormal patterns associated with cancer.

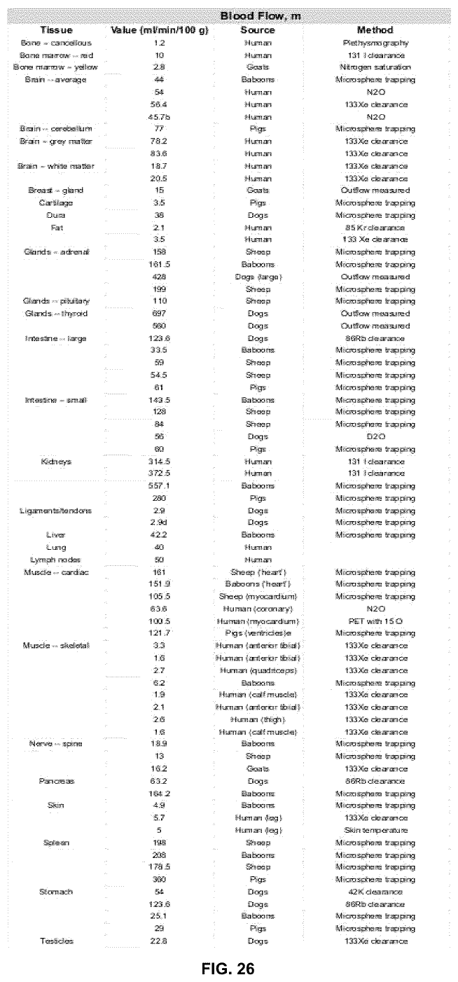

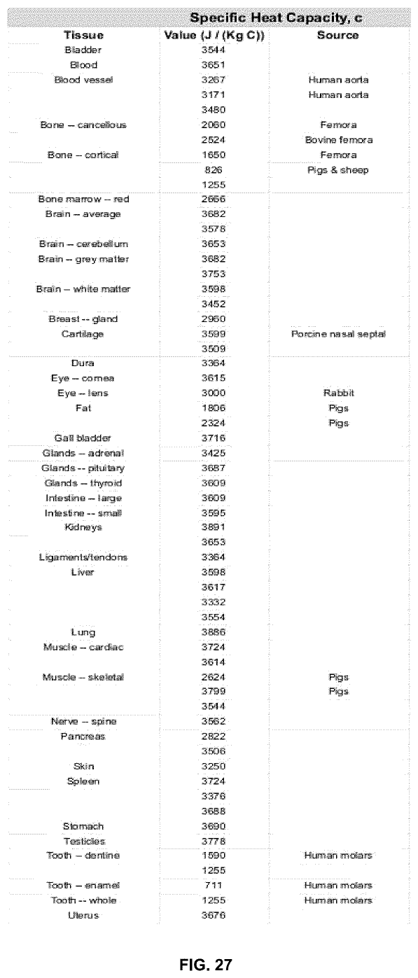

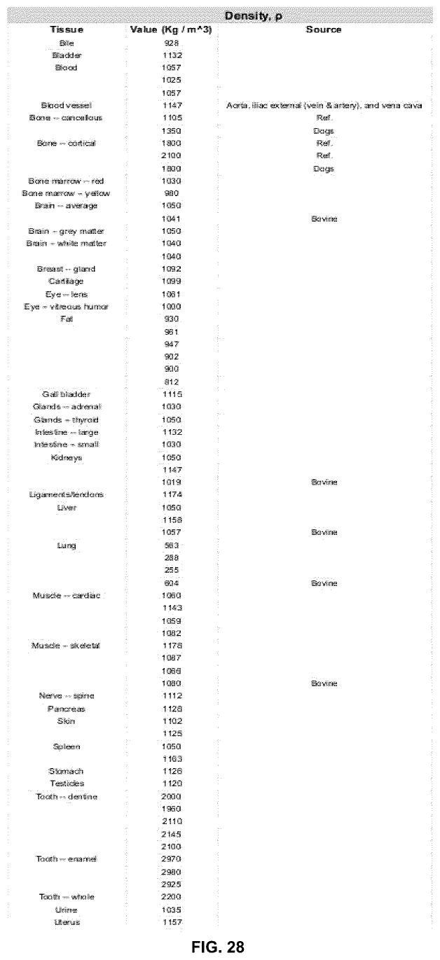

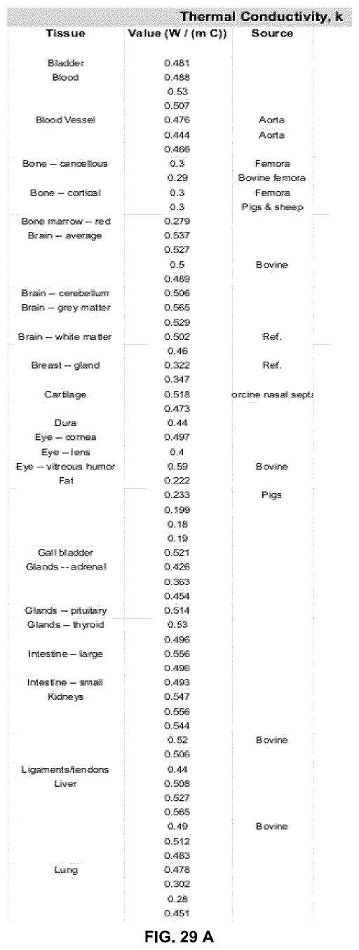

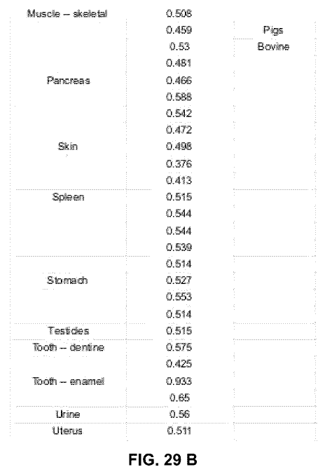

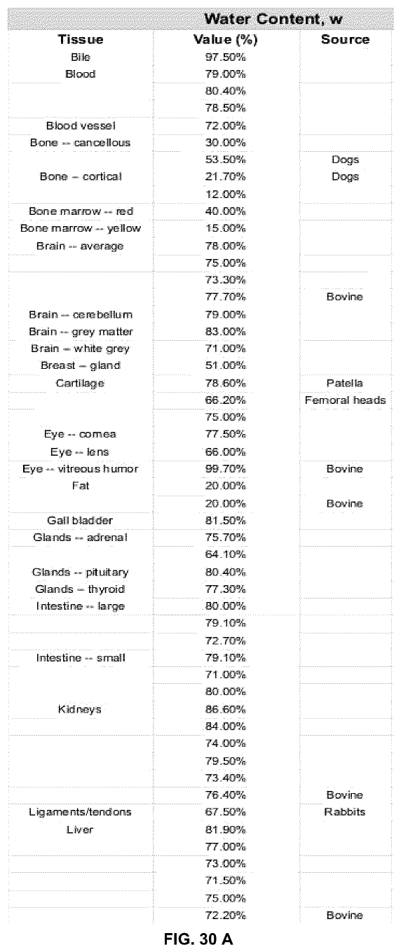

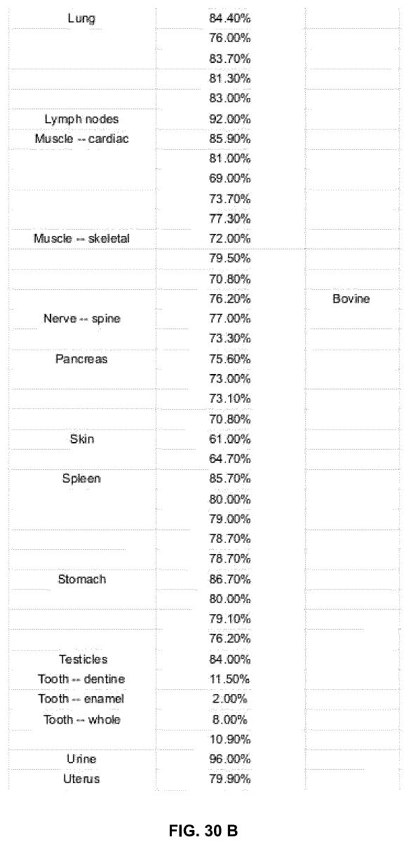

Temperature distribution across healthy and cancerous tissue varies due to increased vascularization of the tumor. Therefore the mechanical stress-strain of the tumor tissue is higher in comparison to a healthy tissue. Materials subjected to stress-strain change their behaviors based on the percentage of the stress they are under and for how long the stress is prolonged. Therefore knowing tissue parameters of the human skin and the composition of the organs from literature is of great importance to understand the mechanical stress of the tissue. Parameters found in literature such as a) blood flow, b) specific heat capacity for each tissues, c) density of the tissues, d) thermal conductivity and e) water content as shown in FIG. 26, FIG. 27, FIG. 28, FIG. 29 A, FIG. 29 B, FIG. 30A and FIG. 30 B altogether will provide stress-strain information of the tissue when invaded by tumor. To be more precise it will be possible to build a stress-strain map based on the information provided by the vibrational intensity and the mechanical parameters introduced above.

The stress-strain map will therefore display the magnitude as color in a two-dimensional matrix. Each dimension represents a category of trait and the color represents the magnitude of specific measurement on the combined traits from each of the two categories. One dimension represents the length of the scanned area and the other dimension represents the width of the scanned area, and the value measured is the depth.

This map would show how stress-strain is distributed across different locations of the scanned area under investigation and particularly on the surface of every square discretized area. From a stress-strain map it is possible to create a stiffness map. The variation in color may be by hue or intensity, giving obvious visual cues to the user about how the phenomenon varies over space. Also it would relate to the specific vibration.

Phantom material made of polymeric gels are structurally soft and flexible materials mainly used for mimicking tissue and organs. Due to their shapeability it is possible to manufacture gels of different shapes that can deform significantly. Natural polymers used for developing piezoelectric material include gelatine, collagen, agarose and cellulose just to name a few. Important examples of synthetic piezoelectric polymers used in hydrogel preparation include polyglycerol, poly acrylic acid and additional synthetic polymers.

Piezoelectric polymer gels can therefore show important mechanical features such as mechanical strength, viscoelasticity, stability and bending, but also electronic features such as current generation when subjected to an external stimulus such as a mechanical stress. An example would be the production of electric potential when a phantom material is subjected to mechanical, impulsive or continuous external stress.

Therefore it is possible to correlate the surface vibrational response mentioned earlier with surface charge of the polymer (or the skin). More specifically it is known in the art that human skin has a permanent electric dipole, therefore voltage responses to pressure pulses caused by an external stimulus (such as a Q-Switched laser) are piezoelectric. Additionally, there have been several studies on cancer cells both in the oncology and diagnostic field showing that tumor cells are characterized by a negative charge of the cell surface. Therefore, as an example, we can imagine a very common breast cancer that typically starts in the epithelial cells of the breast: a breast carcinoma.

As mentioned, cancer cell surface is characterized by negative charge of the cell surface. The glycolytic area surrounding the cancer host interface is also characterized by a negatively-charged area. On the other hand, human epidermis is positively charged, therefore as soon as an external stimulus is introducing a mechanical stress we should see a negative surface charge related to the area where the cancer is localized, while we may observe a positive surface charge related to the area where there is no cancer. Therefore as an example, we can visualize a contour map with the positively or negatively charged areas.

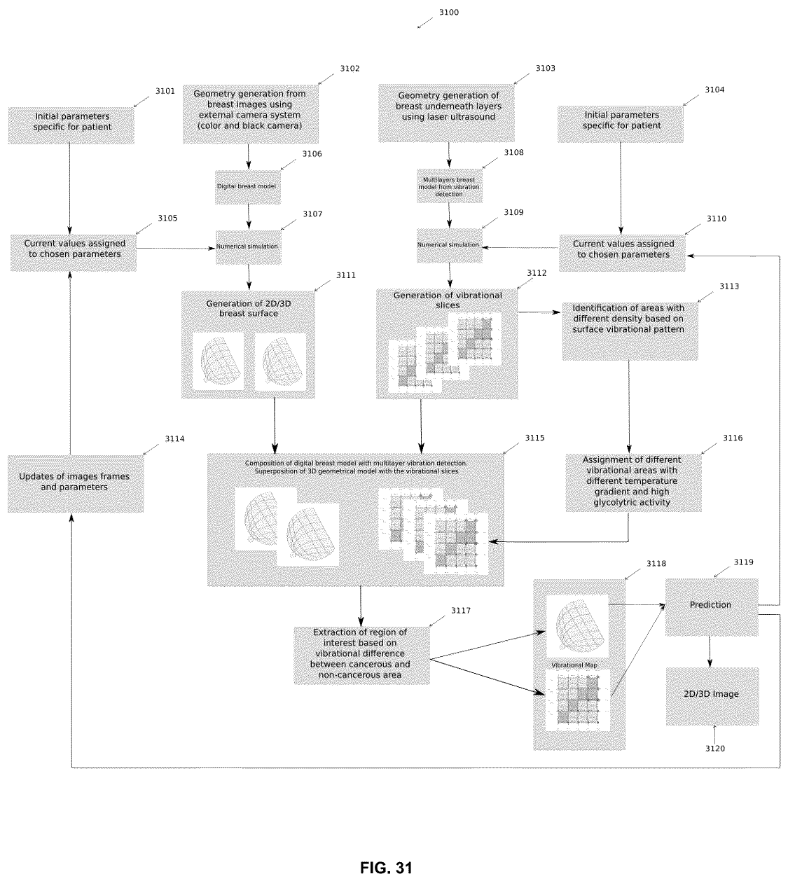

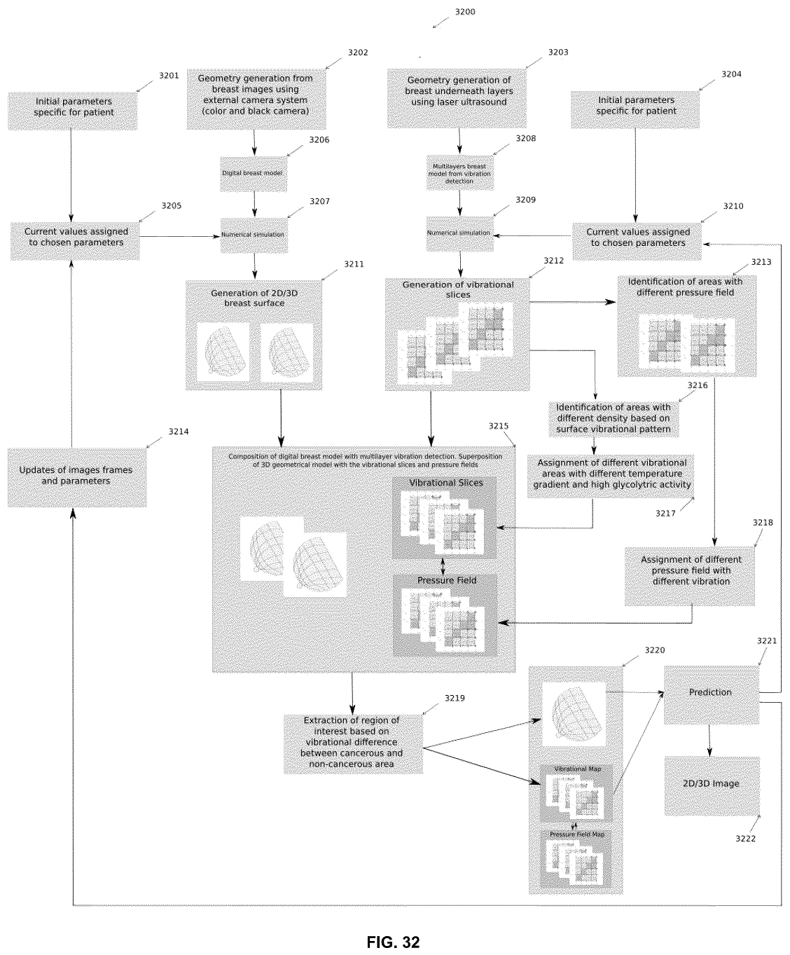

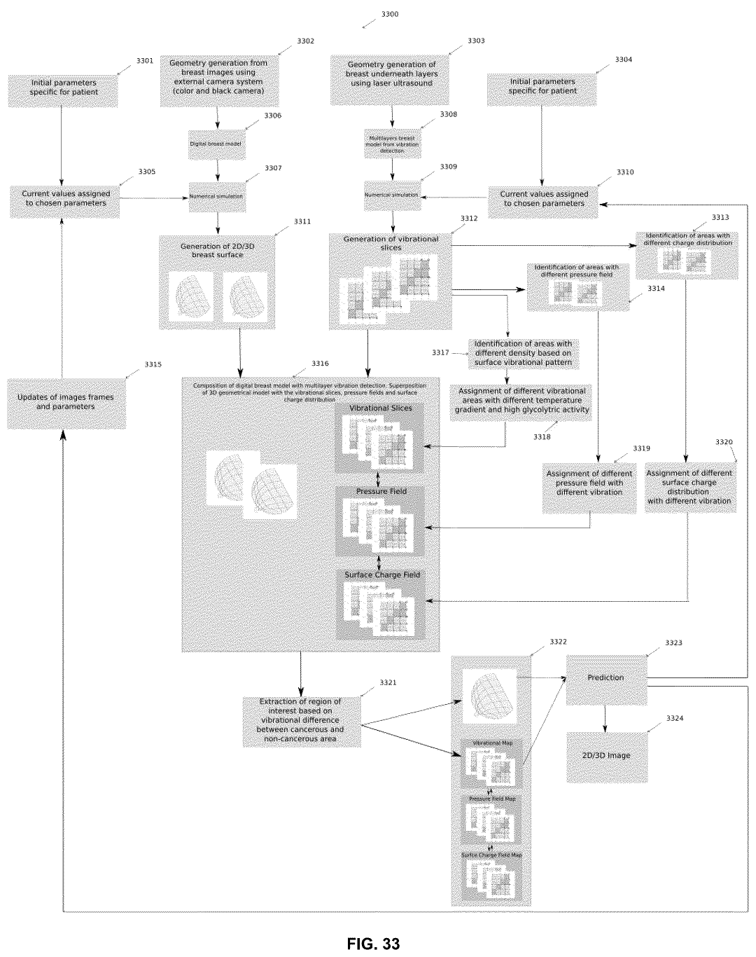

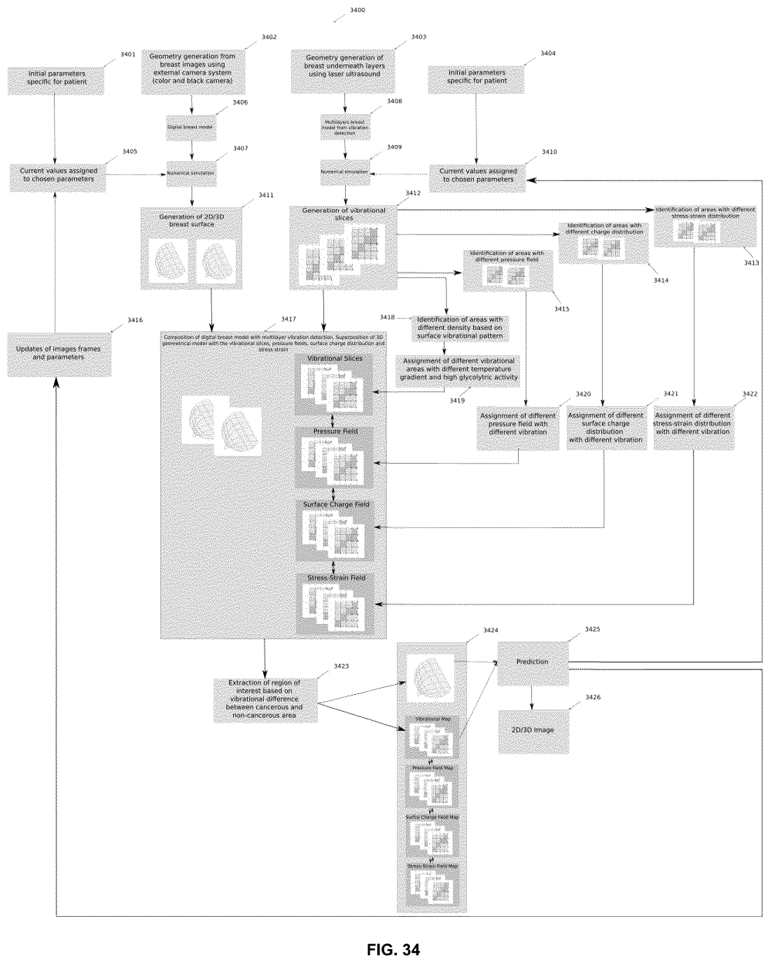

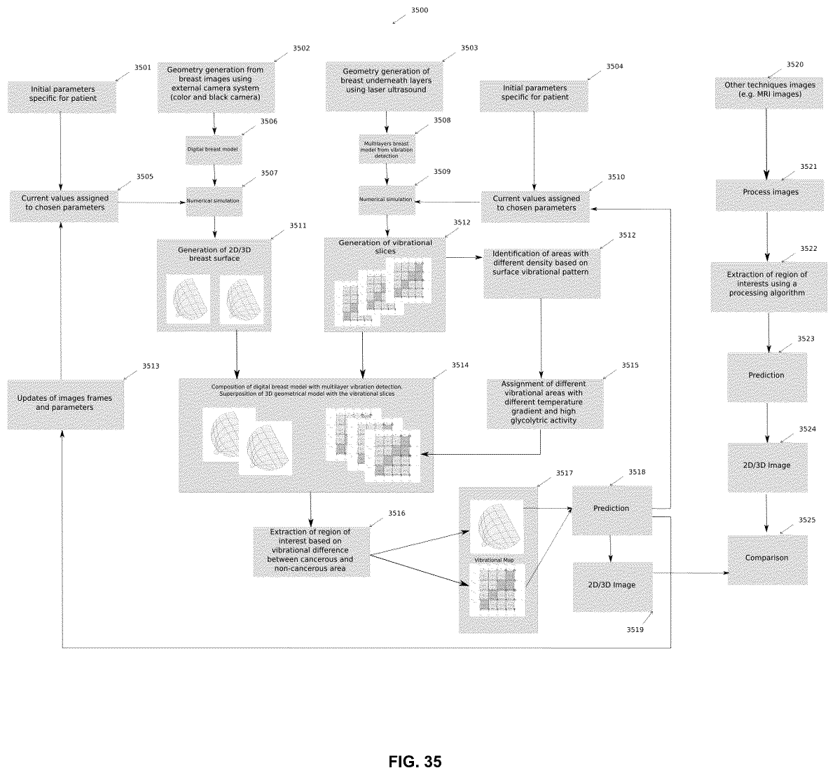

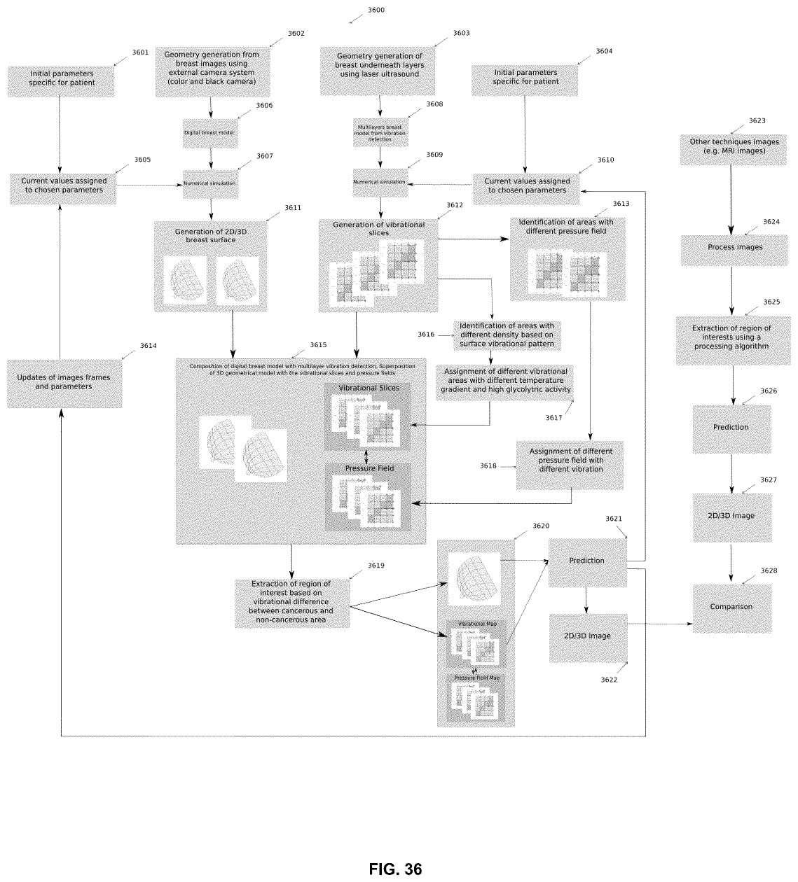

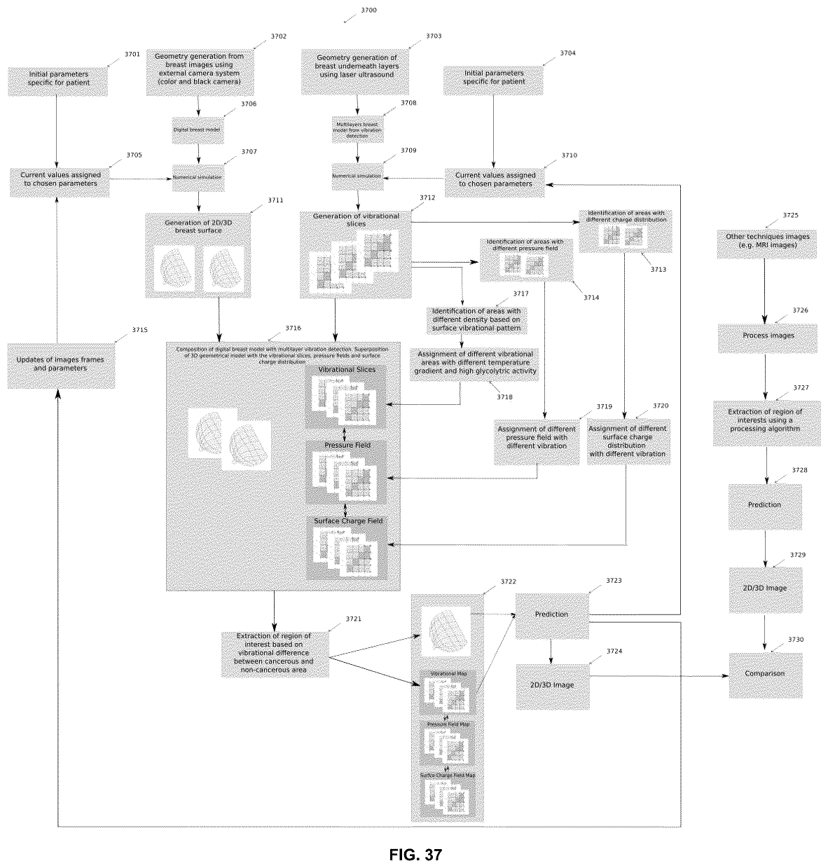

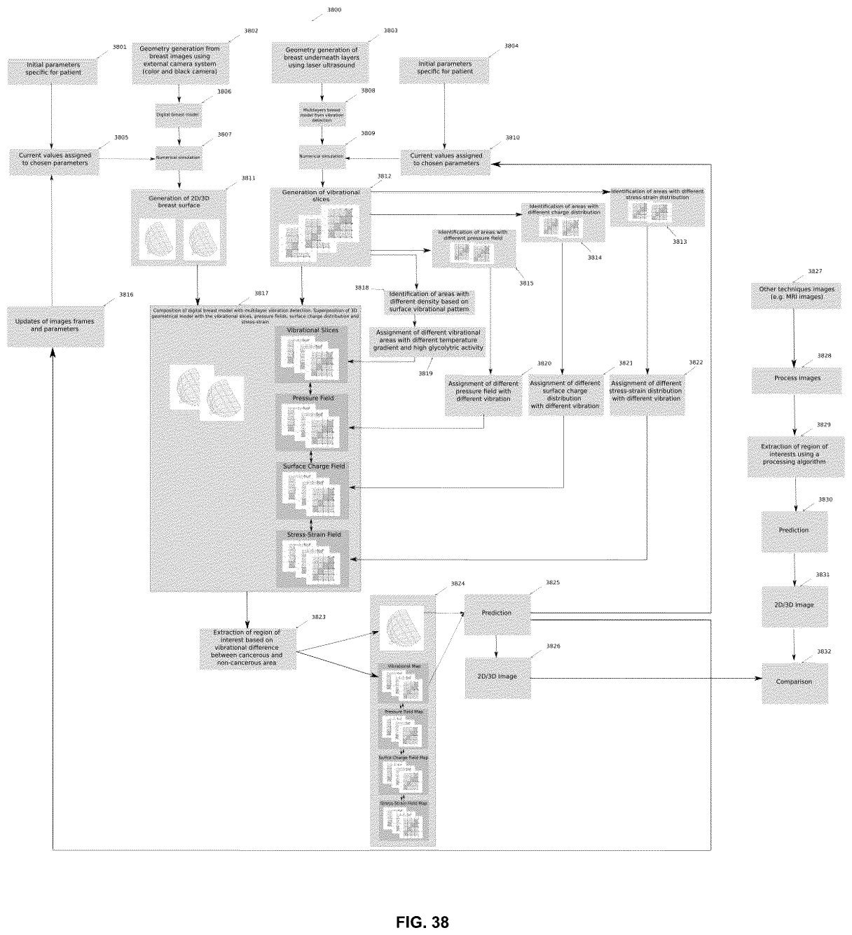

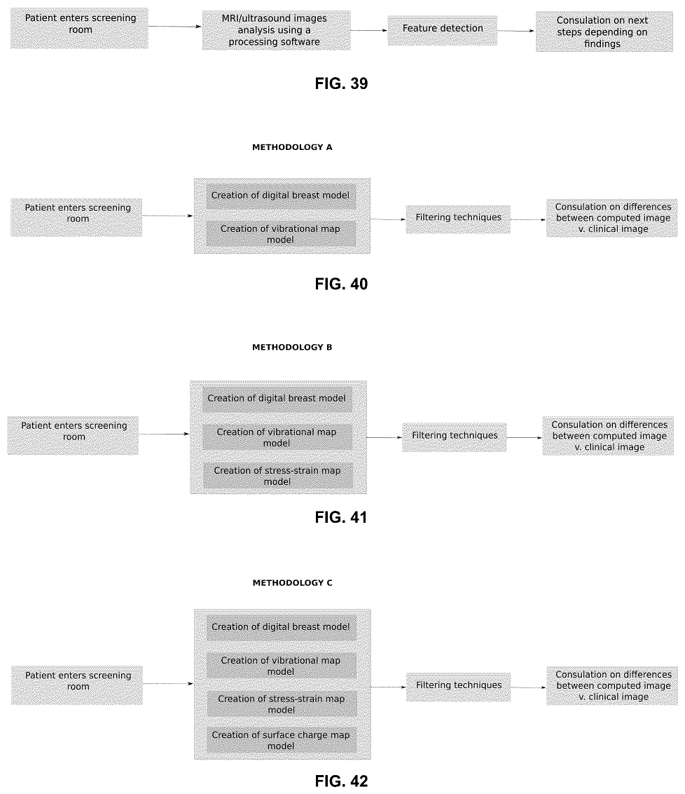

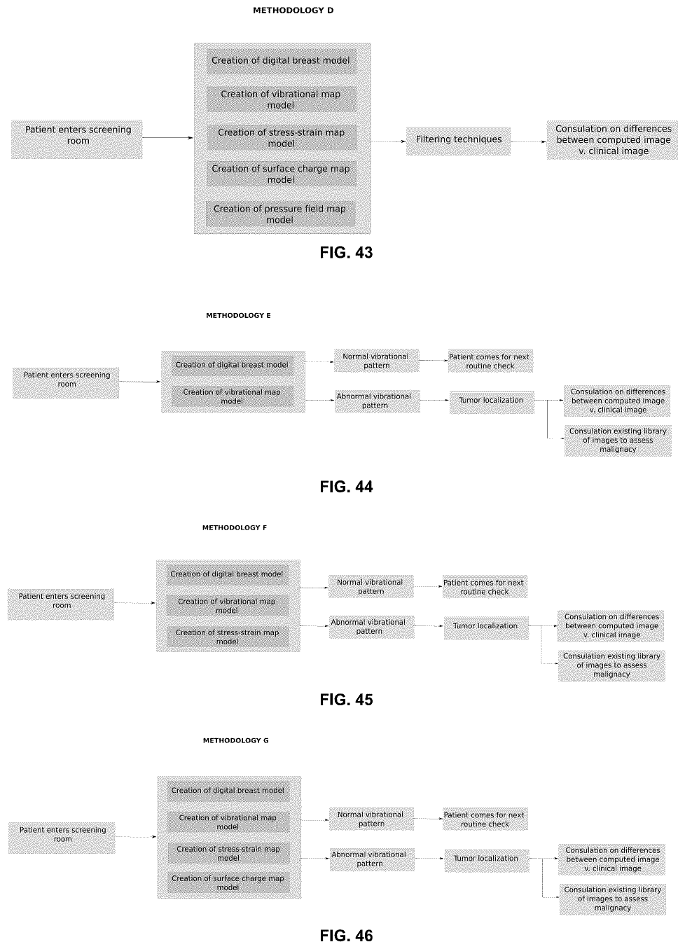

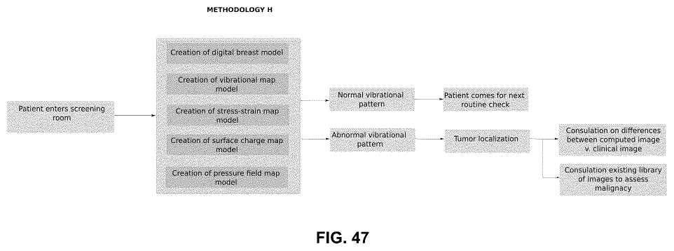

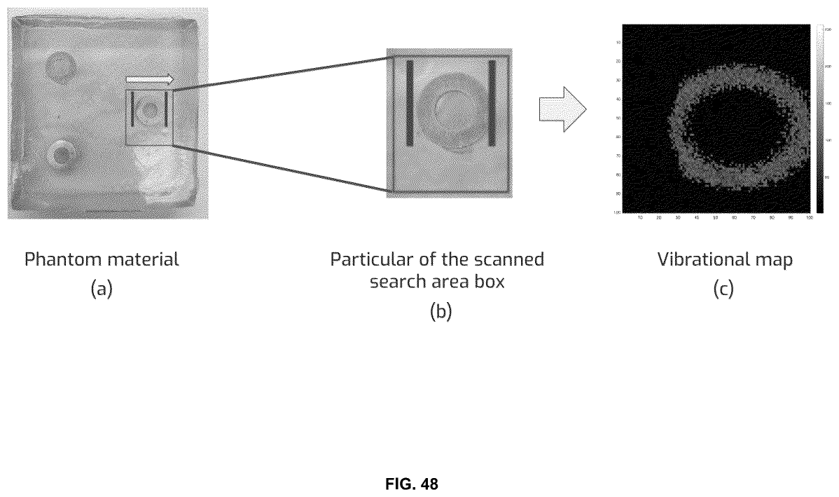

Methodologies listed in this patent are referred to FIG. 31, FIG. 32, FIG. 33, FIG. 34 and by way of comparing the current methodology with known techniques such as MRI or regular ultrasound, additional flowcharts are shown in FIG. 35, FIG. 36, FIG. 37, FIG. 38. Therefore, different methodologies correlated with examples are presented in this patent to analyze regions of interest. Method A in FIG. 31 explains how to generate a digital model of a 2D/3D breast using stereometry and combine it with a vibrational map. Method B in FIG. 32 explains how to generate a digital model of a 2D/3D breast using stereometry and combine it with a vibrational map and a pressure field map. Method C in FIG. 33 explains how to generate a digital model of a 2D/3D breast using stercometry and combine it with a vibrational map, a pressure field map and a surface charge map. Method D in FIG. 34 explains how to generate a digital model of a 2D/3D breast using stereometry and combine it with a vibrational map, a pressure field map, a surface charge map and a stress-strain map. The same techniques explained on methods A, B, C and D are compared to a regular MRI or ultrasound well known imaging technique by way of comparison only. The flowcharts detailed in FIG. 35, FIG. 36, FIG. 37, FIG. 38 are informative only and to show how and when the comparison happens using a digital library of existing images or a database of images. Flowcharts on FIG. 40, FIG. 41, FIG. 42, FIG. 43 are meant to explain in simpler ways the methodologies explained on flowcharts FIG. 31, FIG. 32, FIG. 33, FIG. 34. Similarly, Flowcharts on FIG. 44, FIG. 45, FIG. 46, FIG. 47 are meant to explain in simpler ways the methodologies explained on flowcharts FIG. 35, FIG. 36, FIG. 37, FIG. 38.

METHOD A: A method for determining breast malignancy within tissue using a vibrational map and a digital model generated from a camera system as shown in flowchart 3100 in FIG. 31. A region of interest is identified by a doctor as a region of the breast where additional analysis is necessary based on suspected presence of cancer due to various reasons such as a) family history, b) genetic, c) presence of BRCA1 or BRCA2 or both BRCA1 and BRCA2 mutation and d) visual inspection. These are also considered initial patient parameters as in 3101. For each of the parameters the doctors will provide current values as shown on 3105. Tumors within breast tissue (and more in general within tissues) are characterized by change in density in the affected area, greater amount of heat propagation in the tissue and surrounding tissue due to increased vascularization of the tumor and the growing stage.

After the patient sits standstill on a sitting device such as a chair, an image is taken with a camera system (two cameras—one camera taking black/white images and one camera taking colored images) such that the distance between the two cameras is known. The two cameras need to be one next to the other and mounted on a linear platform. In this way it is possible to create stereo-images. A stereo image contains two views of a scene side by side. One of the views is intended for the left eye and the other for the right eye. A stereo pair of images taken is used to find distance. After the doctor selects with a highlighter a specific area on the breast for further analysis.

The doctor can either draw with a highlighter on the skin of the patient four points that when united form a rectangular shape or the camera system is equipped with a graphical user interface where the doctor can digitally choose four points and define a search box. Several images are taken of the breast area defined and a geometric map is created using a numerical simulation based also on the physiological parameters of the breast as shown in 3107 and the final image is produced in 3111.

An ultrasound laser system equipped with a Q-Switched lasers (or two Q-Switched lasers fully synchronized) will move on the search area defined by the doctor and send laser pulses toward the areas defined previously as shown on 3103. The same values provided for the camera system are also provided for the laser analysis as shown on 3104 and 3110. However, the main difference here is that the values are specific for the laser such as: i) pulse rate, ii) pulse energy, iii) pulse width and iiii) repetition rate. As soon as the laser pulse ricochets back to the surface of the skin an external vibrometer will detect and pick that maximum vibrational intensity at that point. The maximum vibrational intensity of that point within the defined box will have specific xyz coordinates that can be projected on a cartesian system visible on a computer device.

While the laser system moves with minimal movements pixel by pixel covering the whole surface of the search area defined by the doctor, the vibrometer system will pick up each vibration detected on the surface. In this way, collecting a whole series of vibrations for each pixel, we are able to create a vibrational map where each pixel has a specific intensity which can be visualized on a cartesian system. The whole image of the search box area is therefore defined as a vibrational map at a specific depth according to the laser energy penetration rate. The vibrational map (or slice) is visible on 3112. Note that by increasing or decreasing the energy of the laser it is possible to reach various depths, therefore obtaining various vibrational maps (or slices). Each time the vibrometer detects a surface vibration it could be from the same tissue or from a different tissue, therefore vibrating at a different intensity.

Regions of interest are identified based on the vibrational map and analyzed for suspected malignancy. Identification of areas with different density based on surface vibration pattern is therefore analyzed as shown on 3113. Each vibrational pattern detected is assigned with a different color gradient as shown on box 3116.

Assuming the patient's skin is not treated with any specific ultrasonic gel, water or lubricants, the surface emissivity is uniform therefore the vibrational intensity detected by the vibrometer is representative of the vibrational field. Care is therefore taken to avoid the application of skin lotion, creams, gel or lubricants because those may change the emissivity and are therefore not encouraged before imaging.

At this point having both the digitally created model of the breast surface using stereometry in 3111 and the vibrational slice(es) at different depth(s) as in 3112, the doctor is ready for the superposition (or overlapping) of the digital model with the vibrational intensity map(s) as shown on 3115. At this point the doctor is ready to analyze for any specific region of interest and see if there is any presence of cancerous vs non-cancerous tissue as shown on 3117. At this point the two models 3118 are the input for a prediction algorithm 3119 that, based on those inputs, may foresee additional vibrational layers.

The prediction step is made by the use of a particle filter (PF). The algorithm may be used in different scenarios. If there is no additional need to create more vibrational layers, than a final 2D or 3D image is created as shown on 3120, otherwise if there is need to go deeper within the tissue and create more vibrational maps at different layers, than the new vibrational intensity values obtained are passed and updated to the box 3110 so that a new cycle can start until the curing physician is satisfied.

Boxes 3106 and 3108 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD B: A method for determining breast malignancy within tissue using a vibrational map, a pressure field map and a digital model generated from a camera system as shown in flowchart 3200 in FIG. 32. A region of interest is identified by a doctor as a region of the breast where additional analysis is necessary based on suspected presence of cancer due to various reasons such as a) family history, b) genetic, c) presence of BRCA1 or BRCA2 or both BRCA1 and BRCA2 mutation and d) visual inspection. These are also considered initial patient parameters as in 3201. For each of the parameters the doctors will provide current values as shown in 3205. Tumors within breast tissue (and more in general within tissues) are characterized by change in density in the affected area, greater amount of heat propagation in the tissue and surrounding tissue due to increased vascularization of the tumor and the growing stage.

After the patient sits standstill on a sitting device such as a chair, an image is taken with a camera system (two cameras-one camera taking black/white images and one camera taking colored images) such that the distance between the two cameras is known. The two cameras need to be one next to the other and mounted on a linear platform. In this way it is possible to create stereo-images. A stereo image contains two views of a scene side by side. One of the views is intended for the left eye and the other for the right eye. A stereo pair of images taken is used to find distance. After the doctor selects with a highlighter a specific area on the breast for further analysis.

The doctor can either draw with a highlighter on the skin of the patient four points that when united form a rectangular shape or the camera system is equipped with a graphical user interface where the doctor can digitally choose four points and define a search box. Several images are taken of the breast area defined and a geometric map is created using a numerical simulation based also on the physiological parameters of the breast as shown in 3207 and the final image is produced in 3211.

An ultrasound laser system equipped with a Q-Switched lasers (or two Q-Switched lasers fully synchronized) will move on the search area defined by the doctor and send laser pulses toward the areas defined previously as shown on 3203. The same values provided for the camera system are also provided for the laser analysis as shown on 3204 and 3210. However, the main difference here is that the values are specific for the laser such as: i) pulse rate, ii) pulse energy, iii) pulse width and iiii) repetition rate. As soon as the laser pulse ricochets back to the surface of the skin an external vibrometer will detect and pick that maximum vibrational intensity at that point. The maximum vibrational intensity of that point within the defined box will have specific xyz coordinates that can be projected on a cartesian system visible on a computer device.

While the laser system moves with minimal movements pixel by pixel covering the whole surface of the search area defined by the doctor, the vibrometer system will pick up each vibration detected on the surface. In this way, collecting a whole series of vibrations for each pixel, we are able to create a vibrational map where each pixel has a specific intensity which can be visualized on a cartesian system. The whole image of the search box area is therefore defined as a vibrational map at a specific depth according to the laser energy penetration rate. The vibrational map (or slice) is visible on 3212 and the pressure field map is visible on 3213. Note that by increasing or decreasing the energy of the laser it is possible to reach various depths, therefore obtaining various vibrational maps (or slices). Each time the vibrometer detects a surface vibration it could be from the same tissue or from a different tissue, therefore vibrating at a different intensity.

Regions of interest are identified based both on the vibrational map and the pressure field map and analyzed for suspected malignancy. Identification of areas with different density based on surface vibration pattern and pressure filed pattern is therefore analyzed as shown on 3216 and 3218. Each vibrational pattern detected is assigned with a different color gradient as shown on box 3216 and 3217. Each pressure field presenting a different pressure value at that specific point is also assigned with a different color gradient as shown on box 3218.

Assuming the patient's skin is not treated with any specific ultrasonic gel, water or lubricants, the surface emissivity is uniform therefore the vibrational intensity detected by the vibrometer is representative of the vibrational field. Care is therefore taken to avoid the application of skin lotion, creams, gel or lubricants because those may change the emissivity and are therefore not encouraged before imaging.

At this point having: a) the digitally created model of the breast surface using stereometry in 3211, b) the vibrational slice(es) at different depth(s) as in 3212 and c) the pressure field map(s) at different depth(s) as in 3213, the doctor is ready for the superposition (or overlapping) of the digital model with the vibrational intensity map(s) and the pressure filed(s) map(s) as shown on 3215. At this point the doctor is ready to analyze for any specific region of interest and see if there is any presence of cancerous vs non-cancerous tissue as shown on 3219. At this point the three models 3220 are the input for a prediction algorithm 3221 that, based on those inputs, may foresee additional vibrational and pressure filed layers.

The prediction step is made by the use of a particle filter (PF). The algorithm may be used in different scenarios. If there is no additional need to create more vibrational and pressure filed layers, than a final 2D or 3D image is created as in 3222, otherwise if there is need to go deeper within the tissue and create more vibrational and pressure field maps at different layers, than the new vibrational intensity values and new pressure field values obtained are passed and updated to the box 3210 so that a new cycle can start until the curing physician is satisfied.

Boxes 3206 and 3208 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD C: A method for determining breast malignancy within tissue using a vibrational map, a pressure field map, a surface charge map and a digital model generated from a camera system as shown in flowchart 3300 in FIG. 33. A region of interest is identified by a doctor as a region of the breast where additional analysis is necessary based on suspected presence of cancer due to various reasons such as a) family history, b) genetic, c) presence of BRCA1 or BRCA2 or both BRCA1 and BRCA2 mutation and d) visual inspection. These are also considered initial patient parameters as in 3301. For each of the parameters the doctors will provide current values as shown in 3305. Tumors within breast tissue (and more in general within tissues) are characterized by change in density in the affected area, greater amount of heat propagation in the tissue and surrounding tissue due to increased vascularization of the tumor and the growing stage.

After the patient sits standstill on a sitting device such as a chair, an image is taken with a camera system (two cameras-one camera taking black/white images and one camera taking colored images) such that the distance between the two cameras is known. The two cameras need to be one next to the other and mounted on a linear platform. In this way it is possible to create stereo-images. A stereo image contains two views of a scene side by side. One of the views is intended for the left eye and the other for the right eye. A stereo pair of images taken is used to find distance. After the doctor selects with a highlighter a specific area on the breast for further analysis.

The doctor can either draw with a highlighter on the skin of the patient four points that when united form a rectangular shape or the camera system is equipped with a graphical user interface where the doctor can digitally choose four points and define a search box. Several images are taken of the breast area defined and a geometric map is created as shown in 3311.

An ultrasound laser system equipped with a Q-Switched lasers (or two Q-Switched lasers fully synchronized) will move on the search area defined by the doctor and send laser pulses toward the areas defined previously as shown on 3303. The same values provided for the camera system are also provided for the laser analysis as shown on 3304 and 3310. However, the main difference here is that the values are specific for the laser such as: i) pulse rate, ii) pulse energy, iii) pulse width and iiii) repetition rate. As soon as the laser pulse ricochets back to the surface of the skin an external vibrometer will detect and pick that maximum vibrational intensity at that point. The maximum vibrational intensity of that point within the defined box will have specific xyz coordinates that can be projected on a cartesian system visible on a computer device.

While the laser system moves with minimal movements pixel by pixel covering the whole surface of the search area defined by the doctor, the vibrometer system will pick up each vibration detected on the surface. In this way, collecting a whole series of vibrations for each pixel, we are able to create a vibrational map where each pixel has a specific intensity which can be visualized on a cartesian system. The whole image of the search box area is therefore defined as a vibrational map at a specific depth according to the laser energy penetration rate. The vibrational map (or slice) is visible on 3312, the pressure field map is visible on 3314 and the surface charge map is visible on 3313. Note that by increasing or decreasing the energy of the laser it is possible to reach various depths, therefore obtaining various vibrational maps (or slices). Each time the vibrometer detects a surface vibration it could be from the same tissue or from a different tissue, therefore vibrating at a different intensity.

Regions of interest are identified based both on the vibrational map, pressure field map and surface charge map and analyzed for suspected malignancy. Identification of areas with different density based on surface vibration pattern, pressure filed pattern and surface distribution is therefore analyzed as shown on 3317. Each vibrational pattern detected is assigned with a different color gradient as shown on box 3318. Each pressure field presenting a different pressure value at that specific point is also assigned with a different color gradient as shown on box 3319 and surface charge which can be positively or negatively charged and associated with that depth are also assigned a different gradient as shown on 3320. For the charge map we should see a negative surface charge related to the area where the cancer is localized, while we may observe a positive surface charge related to the area where there is no cancer. We may define positively charged contour areas with a “+” sign, whereas negatively charged areas denoted with a “−” sign. Therefore as an example, we can visualize a contour map with the positively or negatively charged areas or with “+” or “−”.

Assuming the patient's skin is not treated with any specific ultrasonic gel, water or lubricants, the surface emissivity is uniform therefore the vibrational intensity detected by the vibrometer is representative of the vibrational field. Care is therefore taken to avoid the application of skin lotion, creams, gel or lubricants because those may change the emissivity and are therefore not encouraged before imaging. Additionally, this aspect is important not to neutralize or alter the natural electric positive dipole of the human skin. Any skin lotion, creams, gel or lubricants may alter those measurements and charge may not be detected anymore.

At this point having: a) the digitally created model of the breast surface using stereometry in 3311, b) the vibrational slice(es) at different depth(s) as in 3312, c) the pressure field map(s) at different depth(s) as in 3314 and d) the surface charge map(s) at different depth(s) as in 3313, the doctor is ready for the superposition (or overlapping) of the digital model with the vibrational intensity map(s), the pressure filed(s) map(s) and the surface density map(s) as shown on 3316. At this point the doctor is ready to analyze for any specific region of interest and see if there is any presence of cancerous vs non-cancerous tissue as shown on 3321. At this point the four models 3323 are the input for a prediction algorithm 3323 that, based on those inputs, may foresee additional vibrational, pressure fields and surface charge distribution layers.

The prediction step is made by the use of a particle filter (PF). The algorithm may be used in different scenarios. If there is no additional need to create more vibrational, pressure field and surface charge layers, than a final 2D or 3D image is created as in 3324, otherwise if there is need to go deeper within the tissue and create more vibrational, pressure field and surface charge maps at different layers, than the new vibrational intensity values, the new pressure field values and the new surface charge values obtained 3323 are passed and updated to the box 3310 so that a new cycle can start until the curing physician is satisfied.

Boxes 3306 and 3308 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD D: A method for determining breast malignancy within tissue using a vibrational map, a pressure field map, a surface charge map, a stress-strain map and a digital model generated from a camera system as shown in flowchart 3400 in FIG. 34. A region of interest is identified by a doctor as a region of the breast where additional analysis is necessary based on suspected presence of cancer due to various reasons such as a) family history, b) genetic, c) presence of BRCA1 or BRCA2 or both BRCA1 and BRCA2 mutation and d) visual inspection. These are also considered initial patient parameters as in 3401. For each of the parameters the doctors will provide current values as shown in 3405. Tumors within breast tissue (and more in general within tissues) are characterized by change in density in the affected area, greater amount of heat propagation in the tissue and surrounding tissue due to increased vascularization of the tumor and the growing stage.

After the patient sits standstill on a sitting device such as a chair, an image is taken with a camera system (two cameras-one camera taking black/white images and one camera taking colored images) such that the distance between the two cameras is known. The two cameras need to be one next to the other and mounted on a linear platform. In this way it is possible to create stereo-images. A stereo image contains two views of a scene side by side. One of the views is intended for the left eye and the other for the right eye. A stereo pair of images taken is used to find distance. After the doctor selects with a highlighter a specific area on the breast for further analysis.

The doctor can either draw with a highlighter on the skin of the patient four points that when united form a rectangular shape or the camera system is equipped with a graphical user interface where the doctor can digitally choose four points and define a search box. Several images are taken of the breast area defined and a geometric map is created as shown in 3411.

An ultrasound laser system equipped with a Q-Switched lasers (or two Q-Switched lasers fully synchronized) will move on the search area defined by the doctor and send laser pulses toward the areas defined previously as shown on 3403. The same values provided for the camera system are also provided for the laser analysis as shown on 3404 and 3410. However, the main difference here is that the values are specific for the laser such as: i) pulse rate, ii) pulse energy, iii) pulse width and iiii) repetition rate. As soon as the laser pulse ricochets back to the surface of the skin an external vibrometer will detect and pick that maximum vibrational intensity at that point. The maximum vibrational intensity of that point within the defined box will have specific xyz coordinates that can be projected on a cartesian system visible on a computer device.

While the laser system moves with minimal movements pixel by pixel covering the whole surface of the search area defined by the doctor, the vibrometer system will pick up each vibration detected on the surface. In this way, collecting a whole series of vibrations for each pixel, we are able to create a vibrational map where each pixel has a specific intensity which can be visualized on a cartesian system. The whole image of the search box area is therefore defined as a vibrational map at a specific depth according to the laser energy penetration rate. The vibrational map (or slice) is visible on 3412, the pressure field map is visible on 3415, the surface charge map is visible on 3414 and the stress-strain map is visible on 3413. Note that by increasing or decreasing the energy of the laser it is possible to reach various depths, therefore obtaining various vibrational maps (or slices). Each time the vibrometer detects a surface vibration it could be from the same tissue or from a different tissue, therefore vibrating at a different intensity.

Regions of interest are identified based both on the vibrational map, pressure field map, surface charge map and stress-strain map and analyzed for suspected malignancy. Identification of areas with different density based on surface vibration pattern, pressure filed pattern, surface distribution and stress-strain pattern is therefore analyzed as shown on 3418. Each vibrational pattern detected is assigned with a different color gradient as shown on box 3419. Each pressure field presenting a different pressure value at that specific point is also assigned with a different color gradient as shown on box 3420 and surface charge which can be positively or negatively charged and associated with that depth are also assigned a different gradient as shown on 3421. For the charge map we should see a negative surface charge related to the area where the cancer is localized, while we may observe a positive surface charge related to the area where there is no cancer. We may define positively charged contour areas with a “+” sign, whereas negatively charged areas denoted with a “−” sign. Therefore as an example, we can visualize a contour map with the positively or negatively charged areas or with “+” or “−”. Lastly, each stress-strain field presenting a different stress-strain value at that specific point is also assigned with a different color gradient as shown on box 3422.

Assuming the patient's skin is not treated with any specific ultrasonic gel, water or lubricants, the surface emissivity is uniform therefore the vibrational intensity detected by the vibrometer is representative of the vibrational field. Care is therefore taken to avoid the application of skin lotion, creams, gel or lubricants because those may change the emissivity and are therefore not encouraged before imaging. Additionally, this aspect is important not to neutralize or alter the natural electric positive dipole of the human skin. Any skin lotion, creams, gel or lubricants may alter those measurements and charge may not be detected anymore.

At this point having: a) the digitally created model of the breast surface using stercometry in 3411, b) the vibrational slice(es) at different depth(s) as in 3412, c) the pressure field map(s) at different depth(s) as in 3415, d) the surface charge map(s) at different depth(s) as in 3414 and e) the stress-strain field map(s) at different depth(s) as in 3413, the doctor is ready for the superposition (or overlapping) of the digital model with the vibrational intensity map(s), the pressure filed(s) map(s), the surface density map(s) and the stress-strain field(s) map(s) as shown on 3417. At this point the doctor is ready to analyze for any specific region of interest and see if there is any presence of cancerous vs non-cancerous tissue as shown on 3423. At this point the five models 3424 are the input for a prediction algorithm 3425 that, based on those inputs, may foresee additional vibrational, pressure, stress-strain fields and surface charge distribution layers.

The prediction step is made by the use of a particle filter (PF). The algorithm may be used in different scenarios. If there is no additional need to create more vibrational, pressure, stress-strain field and surface charge layers, than a final 2D or 3D image is created as in 3426, otherwise if there is need to go deeper within the tissue and create more vibrational, pressure, stress-strain field and surface charge maps at different layers, than the new vibrational intensity values, the new pressure field values, the new stress-strain field values and the new surface charge values obtained 3425 are passed and updated to the box 3410 so that a new cycle can start until the curing physician is satisfied.

Boxes 3406 and 3408 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

Regarding the flow charts in FIG. 35, FIG. 36, FIG. 37 and FIG. 38 those are the same flowcharts of FIG. 31, FIG. 32, FIG. 33 and FIG. 34 with the exception that it is introduced a comparison parameter using medically accepted technologies such as MRI or ultrasound. The reason is that for every solution presented in FIG. 31, FIG. 32, FIG. 33 and FIG. 34 it is now associated with a comparison method using a digital library or a database of existing images (e.g. MRI or ultrasound images).

METHOD E: Is the same as METHOD A previously described with the introduction of a comparison term 3525. Therefore, after going through all the steps described in METHOD A and having obtained a satisfactory image as in 3120 or 3519, that image has to now be compared to standard imaging technologies such as MRI or ultrasound. Box 3520 shows how the same patient now undergoes MRI or ultrasound imaging for a direct comparison. MRI or ultrasound images are processed using a variety of feature detection and extraction software techniques and a prediction is made based on forecasting techniques (e.g. gradient descent, Newton-Raphson method to name a few) as shown in box 3521, 3522 and 3523. Finally a 2D or 3D image is now obtained as in 3524 and that image can now be compared in 3525 with the one obtained in 3519. If the quality and resolution of the images is comparable then the comparison is successful. If the images obtained in 3519 are lower than current standard of care, then further iteration and updates to refine the images are necessary and the workflow goes back to 3510 until a comparable image is obtained.

Boxes 3506 and 3508 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD F: Is the same as METHOD B previously described with the introduction of a comparison term 3628. Therefore, after going through all the steps described in METHOD B and having obtained a satisfactory image as in 3222 or 3622, that image has to now be compared to standard imaging technologies such as MRI or ultrasound. Box 3623 shows how the same patient now undergoes MRI or ultrasound imaging for a direct comparison. MRI or ultrasound images are processed using a variety of feature detection and extraction software techniques and a prediction is made based on forecasting techniques (e.g. gradient descent, Newton-Raphson method to name a few) as shown in box 3624, 3625 and 3626. Finally a 2D or 3D image is now obtained as in 3627 and that image can now be compared in 3628 with the one obtained in 3622. If the quality and resolution of the images is comparable then the comparison is successful. If the images obtained in 3622 are lower than current standard of care, then further iteration and updates to refine the images are necessary and the workflow goes back to 3610 until a comparable image is obtained.

Boxes 3606 and 3608 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD G: Is the same as METHOD C previously described with the introduction of a comparison term 3730. Therefore, after going through all the steps described in METHOD C and having obtained a satisfactory image as in 3324 or 3724, that image has to now be compared to standard imaging technologies such as MRI or ultrasound. Box 3725 shows how the same patient now undergoes MRI or ultrasound imaging for a direct comparison. MRI or ultrasound images are processed using a variety of feature detection and extraction software techniques and a prediction is made based on forecasting techniques (e.g. gradient descent, Newton-Raphson method to name a few) as shown in box 3726, 3727 and 3728. Finally a 2D or 3D image is now obtained as in 3729 and that image can now be compared in 3730 with the one obtained in 3724. If the quality and resolution of the images is comparable then the comparison is successful. If the images obtained in 3724 are lower than current standard of care, then further iteration and updates to refine the images are necessary and the workflow goes back to 3710 until a comparable image is obtained.

Boxes 3706 and 3708 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

METHOD H: Is the same as METHOD D previously described with the introduction of a comparison term 3832. Therefore, after going through all the steps described in METHOD D and having obtained a satisfactory image as in 3426 or 3826, that image has to now be compared to standard imaging technologies such as MRI or ultrasound. Box 3827 shows how the same patient now undergoes MRI or ultrasound imaging for a direct comparison. MRI or ultrasound images are processed using a variety of feature detection and extraction software techniques and a prediction is made based on forecasting techniques (e.g. gradient descent, Newton-Raphson method to name a few) as shown in box 3828, 3829 and 3838. Finally a 2D or 3D image is now obtained as in 3831 and that image can now be compared in 3832 with the one obtained in 3826. If the quality and resolution of the images is comparable then the comparison is successful. If the images obtained in 3826 are lower than current standard of care, then further iteration and updates to refine the images are necessary and the workflow goes back to 3810 until a comparable image is obtained.

Boxes 3806 and 3808 exist in case the patient goes back for a second time or needs to go back multiple times. Therefore it would just be enough for the curing physician to upload the previous digital breast model and vibration patterns and proceed with the workflow until an image is produced. In this way it would be possible to see any meaningful differences between sessions.

Flowcharts on FIG. 40, FIG. 41, FIG. 42, FIG. 43 are meant to explain in simpler ways the methodologies explained on flowcharts FIG. 31, FIG. 32, FIG. 33, FIG. 34. Similarly, Flowcharts on FIG. 44, FIG. 45, FIG. 46, FIG. 47 are meant to explain in simpler ways the methodologies explained on flowcharts FIG. 35, FIG. 36, FIG. 37, FIG. 38.

Explanation of how the algorithm assigns a specific vibrational intensity to a point: in this section of the patent it is given a detailed explanation of how the vibrational assignment algorithm works.

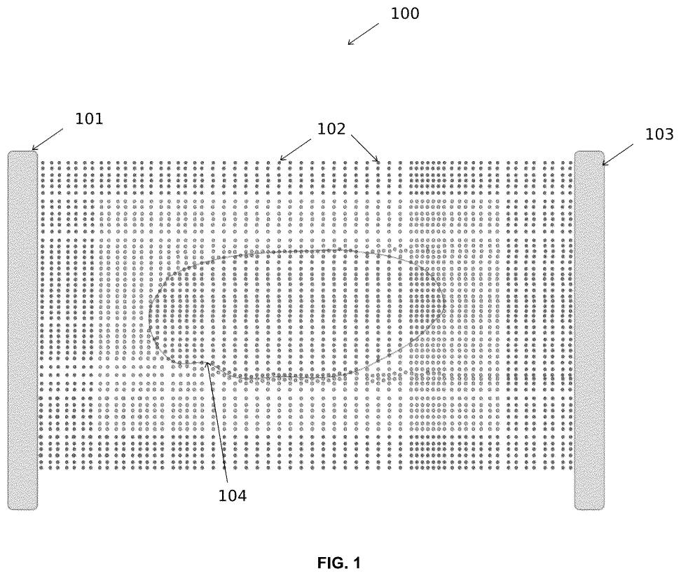

FIG. 1 represents an example of an image taken with a laser ultrasonic technique. Each and every point shown is the maximum vibrational intensity detected by the external vibrometer. Example points are shown on 102 where the indicated points have the same vibrational intensity, in fact they are color coded in the same way. While 104 represents points that have a different vibrational intensity, in fact they are color codes differently to 102. 104 represents a tumorous tissue, while 102 represents a healthy tissue. 101 and 103 in this case are an ideal representation of blood vessels.

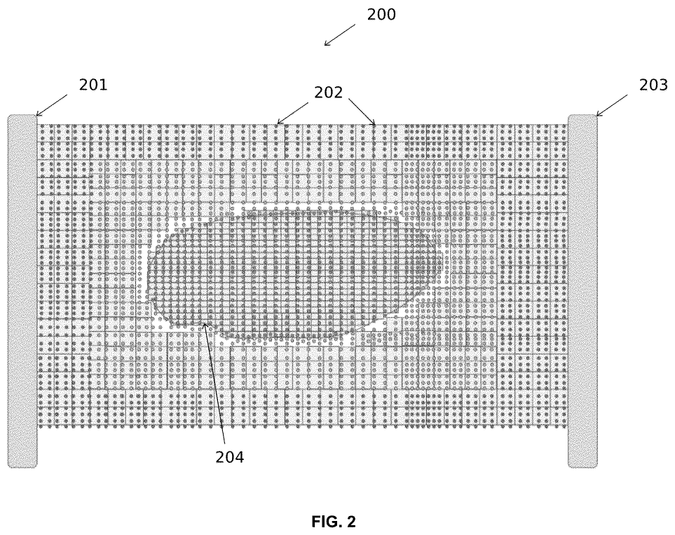

All the points that are color coded in the same way having the same intensity are united in order to form squares (or tiles) of the same length forming a perfect square. On FIG. 2, 202 represents an example of squares of the same intensity while 204 are a representation of squares of a different intensity. Again in this case, the squares in 202 represents a healthy tissue, while the squares in 204 a tumorous tissue. 201 and 203 in this case are an ideal representation of blood vessels.

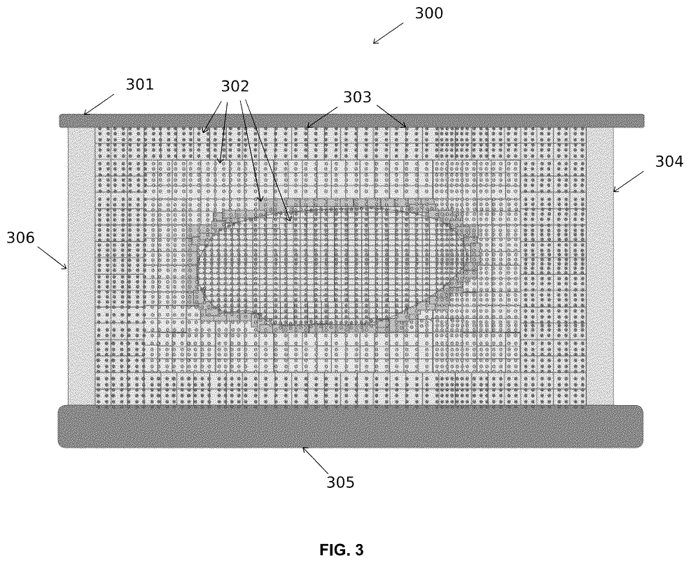

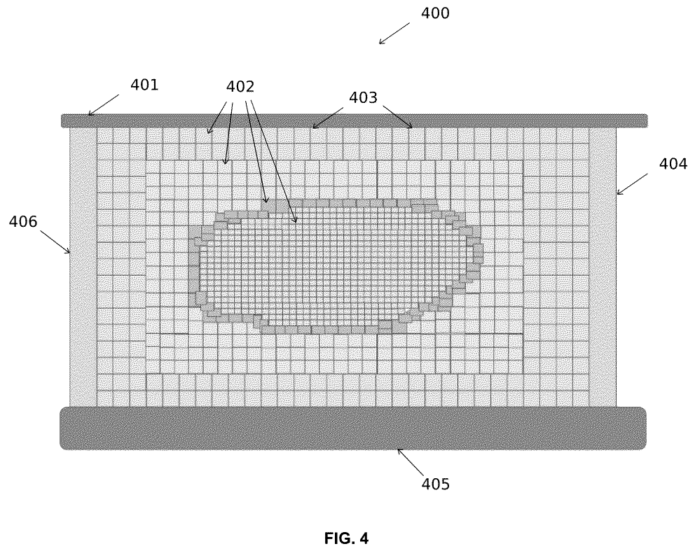

Finally all the squares of the same intensity are therefore given the same color as shown on FIGS. 3. 301, 304, 306 and 305 are an ideal representation of blood vessels surrounding the tumor tissue. 303 is a representation of square color coded the same way, which means that they have the same vibrational intensity. 302 is indicating four different areas, a healthy tissue (closer to the blood vessel), a glycolic tissue which means that the vibrational intensity is different, the tumor host interface which is the immediate border that separates the glycolytic area with the tumor core and finally the tumor core. The image is better appreciated in FIG. 4 where all the points with the same vibrational intensity are averaged altogether and only small perfect squares are depicted. 401, 404, 406 and 405 are an ideal representation of blood vessels surrounding the tumor tissue. 403 is a representation of squares color coded the same way, which means that they have the same vibrational intensity. 402 is indicating four different types of squares, a healthy tissue (closer to the blood vessel), a glycolic tissue which means that the vibrational intensity is different, the tumor host interface which is the immediate border that separates the glycolytic area with the tumor core and finally the tumor core.

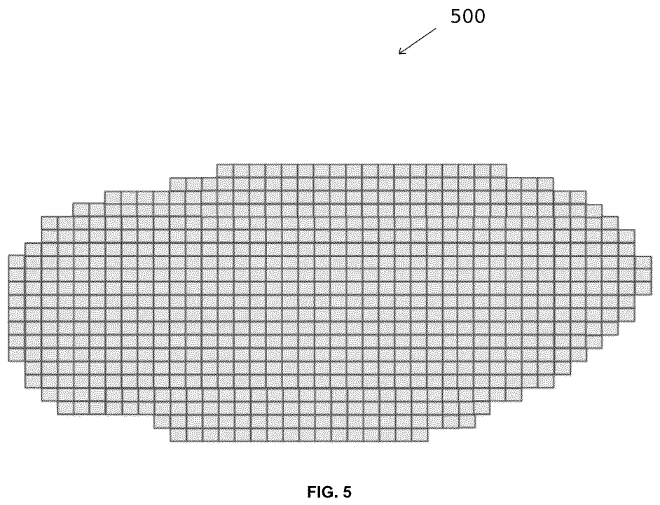

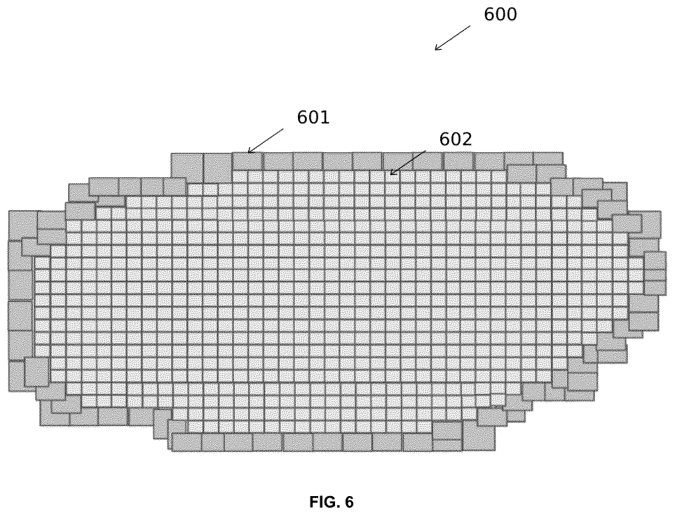

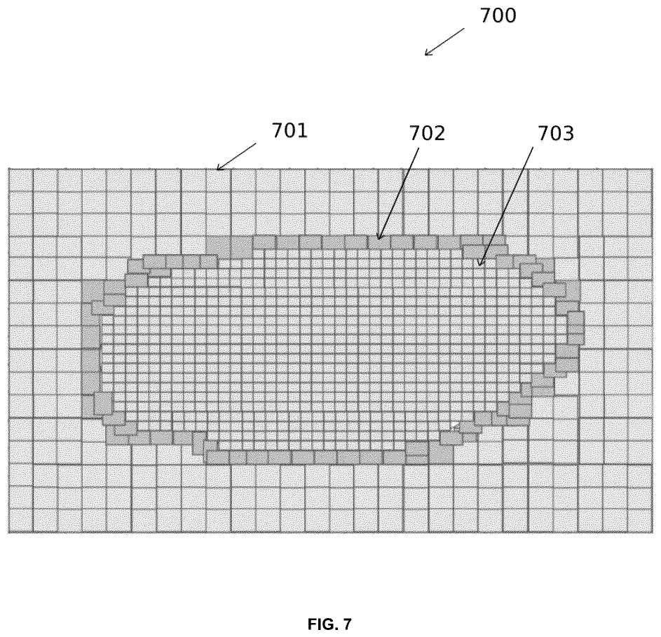

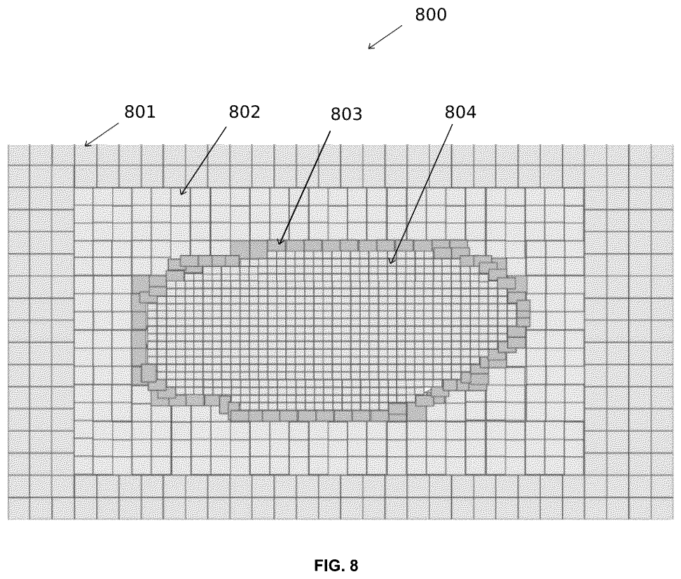

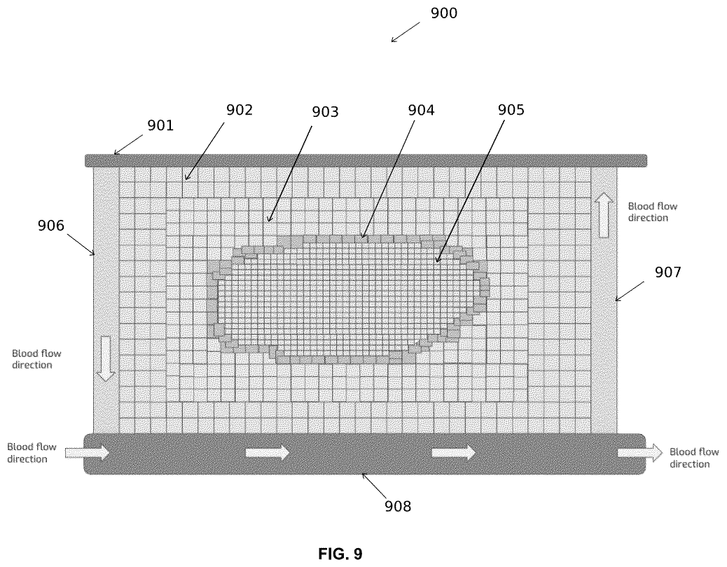

FIG. 5 is a representation of the tumor extracted from FIG. 4. FIG. 6 is a representation of the tumor extracted from FIG. 4 where it is visible the tumor core and immediate border of the tumor host interface. 601 and 602 represent the different vibrational intensities of those locations. FIG. 7 is a representation of the tumor extracted from FIG. 4 where it is visible the tumor core 703, the immediate border of the tumor host interface 702 and the glycolytic area 701. 701, 702 and 703 represent the different vibrational intensities of those locations. FIG. 8 is a representation of the tumor extracted from FIG. 4 where it is visible the tumor core 804, the immediate border of the tumor host interface 803, the glycolytic area 802 and the healthy tissue 801. 801, 802, 803 and 804 represent the different vibrational intensities of those locations. Finally FIG. 9 is the complete representation of the tumor system where 901, 907, 906 and 909 are an ideal representation of blood vessels surrounding the tumor tissue and the arrows represent the circulation direction of the blood flow. 902, 903, 904 and 905 represent the different vibrational intensities of those locations.

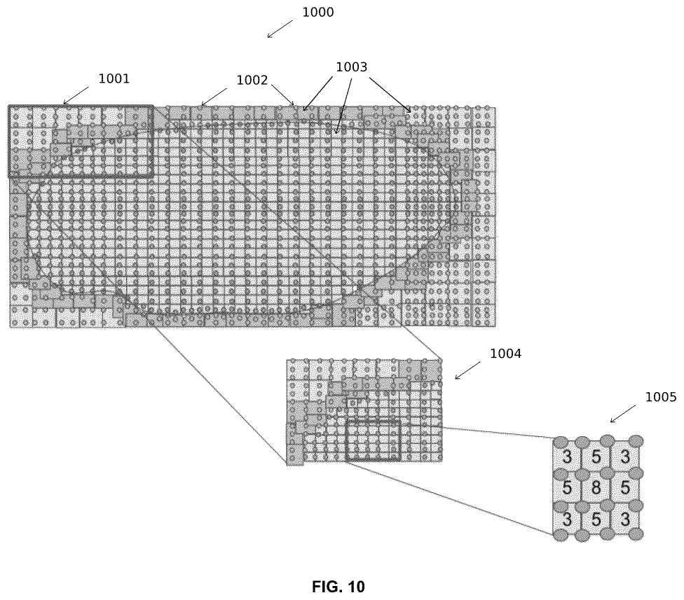

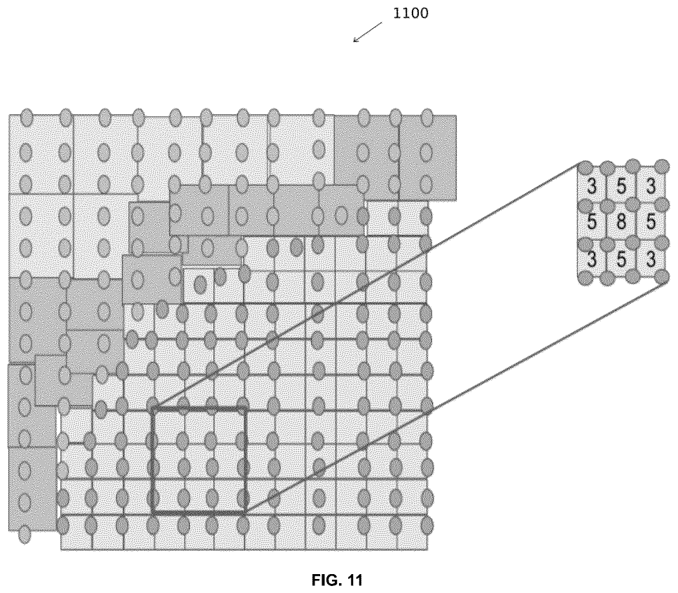

The system depicted in 1000 in FIG. 10 is an enlargement of FIG. 3. A red box 1001 is extracted and shown on 1004. From 1004 another red box is chosen to enlarge the structure of the detected points 1005. 1005 shows how the maximum vibrational point is detected and extracted. As it is visible from this image they are all perfect squares as described above. They also have the same color because they are associated with the same vibrational intensity. FIG. 11 is another enlarged representation of 1004. Next is the core description of the algorithm and how it assigns specific color values associated with similar vibrational intensity(ies).

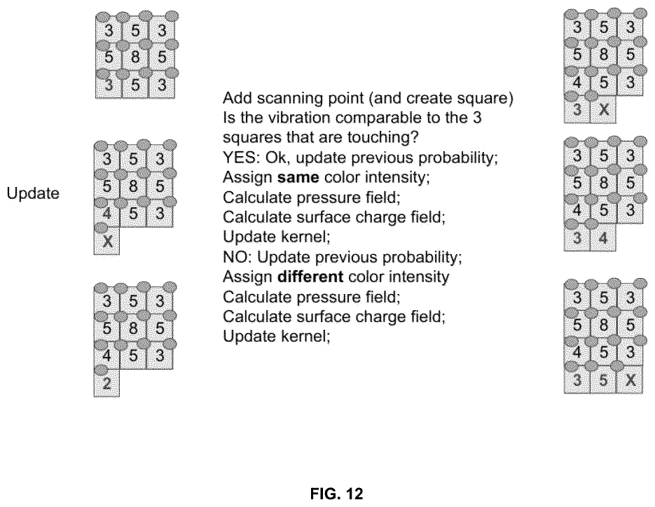

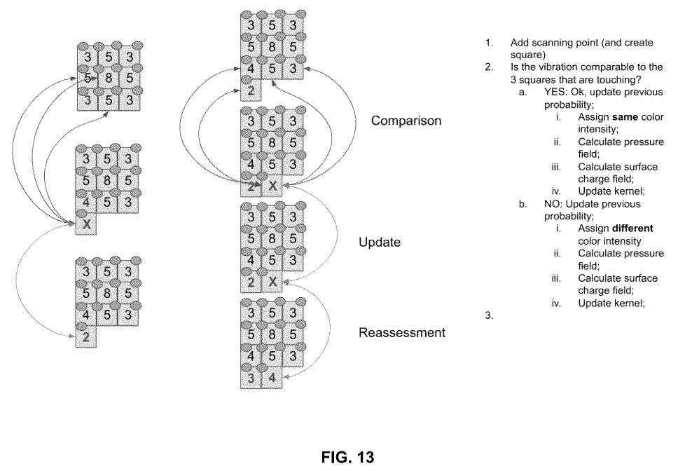

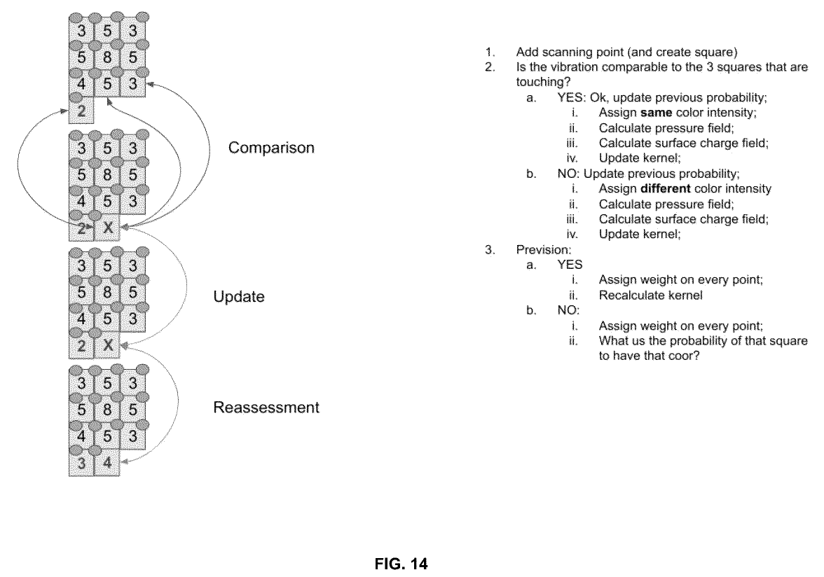

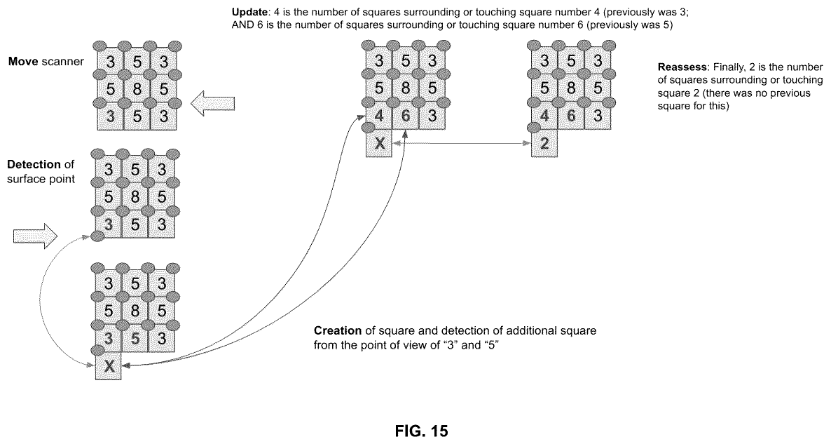

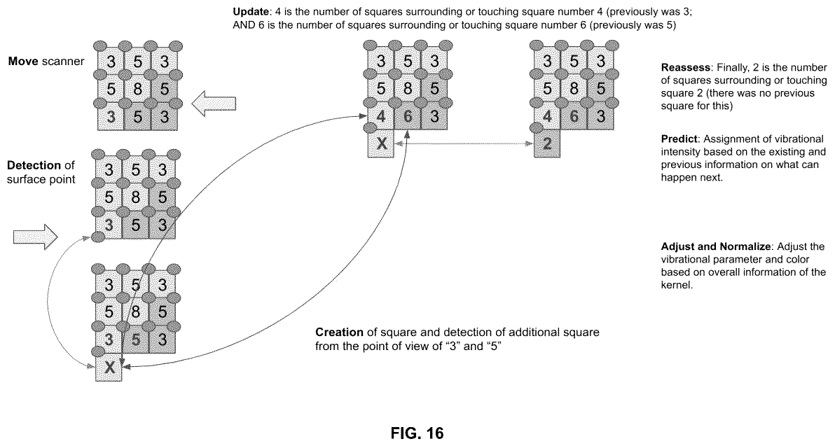

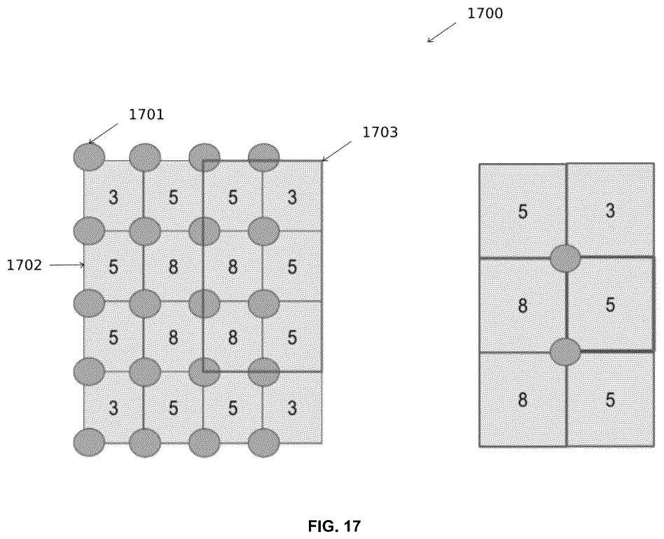

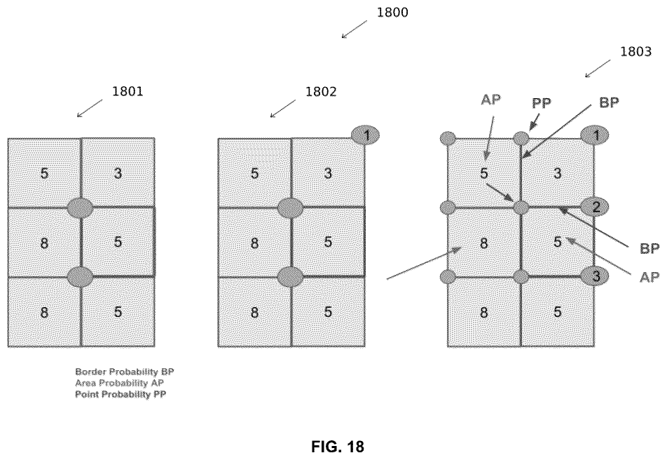

FIG. 12 is the core part of the algorithm. As an example let's take a matrix composed of 3×3 squares. All squares have the same vibrational intensity. In the lower right of the 3×3 squares is where we are going to proceed with the next scanning point, which in fact is added carrying the “X” values. All the values contained within the squares are the number of squares that each square borders with or touches. For example, a value of 3 means that that square is bordering or touching 3 other squares; a value of 5 means that that square is bordering or touching 5 other squares and so on. Since the new square due to the new scanning procedure was just added, we don't know yet what values to give. The algorithm will see that the number of squares that the current “X” touches is 2, therefore the value of “2” is given to the new square. After that the other squares will necessarily be affected by this new square because it introduced new borders and therefore that has to be an update of the current kernel (intended as the new total number of N×N squares) and a reassessment of the value (number) for each square. As soon as the vibration at that specific point is detected then the algorithm compares that vibrational intensities ao all the surrounding squares and assigns a probability that the specific vibration is comparable to the others. FIG. 14 just shows a particular of the newly obtained reassessment value. If the vibrational intensity is indeed comparable to the other points then the same color is provided, otherwise a different color is assigned as shown on FIG. 16. At this stage it will also be possible to operate a prediction step based on the potential vibrational intensity of the next point provided the previous vibrational history of the past points. At this point the cycle repeats until the chosen area is fully scanned. To illustrate more how the algorithm assigns specific probability we can consider FIG. 17 where a bigger kernel of 4×4 is shown and where a red box 1703 is extracted for further analysis. In FIG. 18 we are now ready to introduce 3 different type of probability: a pont probability (PP), a border probability (BP) and an area probability (AP). In this way additional questions that the algorithm has to answer are: how similar is the point probability of point “X” in comparison to other neighboring points? How similar is the border probability of line “Y” in comparison to other neighboring lines? How similar is the area probability of area “Z” in comparison to other neighboring areas?

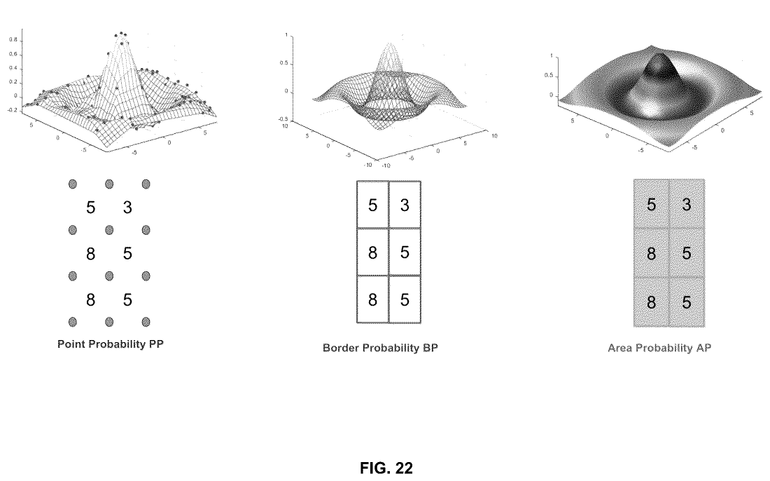

This concept is further illustrated in FIG. 19, FIG. 20 and FIG. 21. FIG. 19 shows a function that can be depicted using points only. If we extract a small portion (a red box) of the function depicted and project this point into a cartesian system, we obtain a total 4×3 points. If we add an additional point due to a new scan, how similar is the point probability of the new point in comparison to other neighboring points? If the new point has a vibrational probability similar to the other then the new point is assigned the same color intensity as the other, otherwise a different color code will be provided. FIG. 20 shows the same function depicted in FIG. 19, with the difference that instead of showing the function via points, the same function is shown with connecting lines. If we extract a small portion (the same red box from FIG. 19) of the function depicted and project these lines into a cartesian system, we obtain a wireframe composed of 6 squares. If we add an additional point due to a new scan, how similar is the border probability of the new line in comparison to other neighboring lines? If the new lines have a vibrational probability similar to the other then the new lines are assigned the same color intensity as the other, otherwise a different color code will be provided. FIG. 21 shows the same function depicted in FIG. 19, with the difference that instead of showing the function via points or bordering lines, the same function is shown with tiles (area of each square). If we extract a small portion (the same red box from FIG. 19 and FIG. 20) of the function depicted and project these areas into a cartesian system, we obtain a tile composed of 6 full tiles (squares). If we add an additional point due to a new scan, how similar is the area probability of the new tile in comparison to other neighboring tiles? If the new tile has a vibrational probability similar to the other then the new tile is assigned the same color intensity as the other, otherwise a different color code will be provided. FIG. 22 shows that the total probability of a tile is the combination of PP, BP and AP. With this detailed explanation now we consider again the red box extracted from 1001 and 1004 which is further enlarged in FIG. 11.

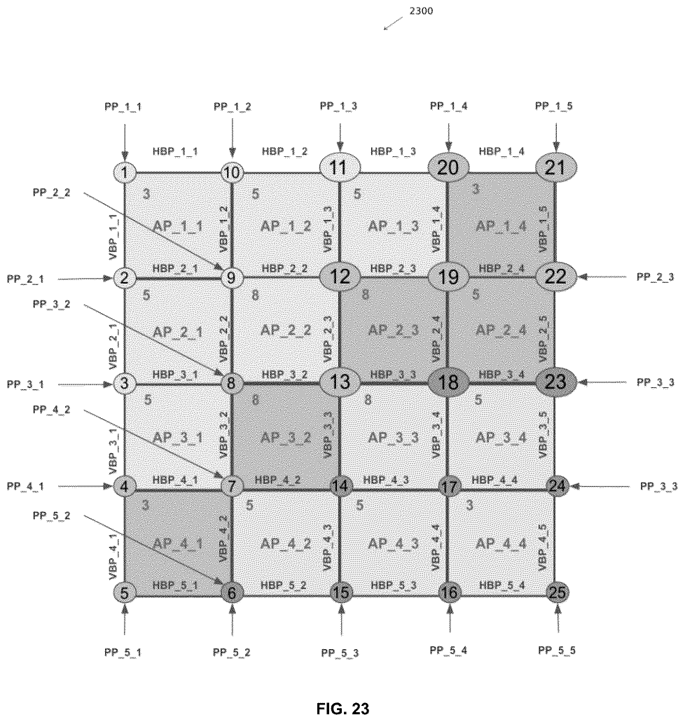

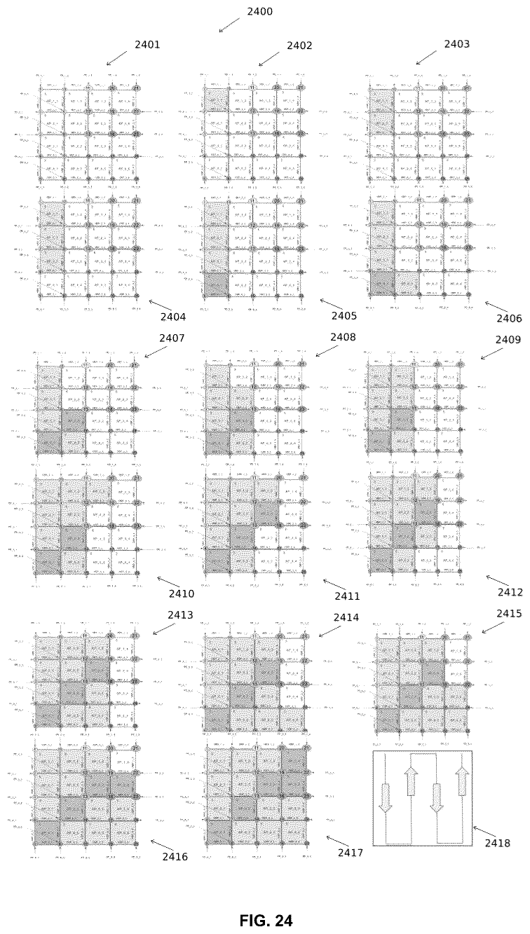

With the same philosophy we can now divide the 1001 box into PP, BP and AP. Border probability is further divided into Horizontal Line Border Probability (HBP) and Vertical Line Border Probability (VBP) as shown in FIG. 23. We can appreciate from FIG. 23 how everything geometrical entities (points and lines) are now assigned a specific probability based on how many objects they are bordering with, how many points they are bordering with and how many areas they are bordering with. The sequential number of points shown in FIG. 23 shows also how the laser system proceeded with the scan. Finally FIG. 24 shows all the processes for how the vibrational intensity is color coded based on all those probabilities. The assignment starts from 2402 up left and proceeds downwards until 2405. After that we move to the right as shown in 2406 and proceed upwards until 2409 and so on until we complete the assignment as in 2417. Finally 2418 depicts in a simplified way the direction of the color coded assignment of the vibrational intensity.

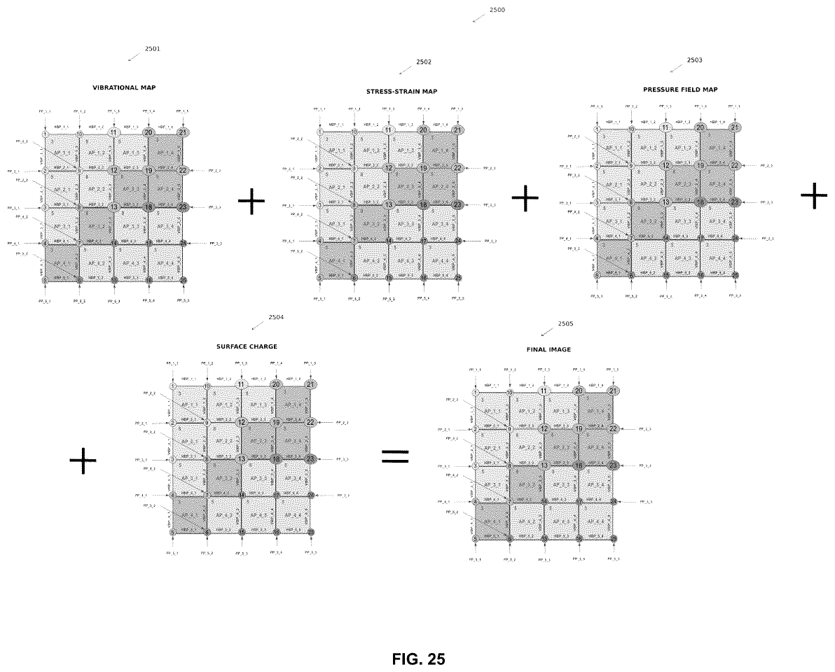

Lastly, FIG. 25 shows how the same algorithm and the same philosophy can be generalized to extract stress-strain maps 2502, pressure field maps 2503, surface charge maps 2504. Therefore the final combined image is shown in 2505. The case portrayed on FIG. 25 carries the same color intensities for all the maps, this is to demonstrate that all the images can be superposed (overlapped) on each other for correspondence and to prove correctness of the probabilities assigned.

The disclosures in this patent will be further illustrated and described with reference to specific examples. It is understood that these examples are given by way of illustration and are not meant to limit the disclosures or the claims to follow.

### Example 1—Method A: Vibrational Maps

In FIG. 48 it is shown a phantom material mimicking a slice of the human breast with the presence of three different targets (which in this case are an ideal representation of a tumor). The middle enlarged box (b) is an example of the part of the phantom (or the body) that will be subjected to scan for suspected malignancy. After proceeding with laser ultrasound techniques the vibrational image obtained is shown in (c).

### Example 2—Method B: Vibrational and Pressure Field Maps

FIG. 49 represents both the vibrational intensity map (a) and from the mechanical vibration is it possible to extract the pressure field response shown in (b). The image shown is discretized with the algorithm described previously and at each pixel is associated a specific color. The color, as also explained in the algorithm section, assigns a specific color based on a similar stimulus (e.g. vibration pattern, pressure pattern) and the two images can now be subjected to image registration. This means that when the two images are overlapped on top of each other they will give two-fold information on vibrational intensity, which can also be correlated to pressure response in that point (or pixel).

### Example 3—Method C: Vibrational, Pressure Field and Surface Charge Map

FIG. 50 represents both the vibrational intensity map (a) from the mechanical vibration and from the vibrational map it is possible to extract both the pressure field response shown in (b) and the surface charge shown in (c). The image shown in (b) is discretized with the algorithm described previously and at each pixel is associated a specific color. The color, as also explained in the algorithm section, assigns a specific color based on a similar stimulus (e.g. vibration pattern, pressure pattern or surface charge) and the three images can now be subjected to image registration. This means that when the three images are overlapped on top of each other they will give three-fold information on vibrational intensity, which can also be correlated to pressure response in that point (or pixel) and surface charge in that point (or pixel).

### Example 4-Method D: Vibrational, Pressure Field, Surface Charge and Stress-Strain Map. Altogether this Forms the VSPC Map

FIG. 51 represents both the vibrational intensity map (a) from the mechanical vibration and from the vibrational map it is possible to extract both the pressure field response shown in (b) and the surface charge shown in (c) and the stress-strain shown in (d). The image shown in (b) is discretized with the algorithm described previously and at each pixel is associated a specific color. The color, as also explained in the algorithm section, assigns a specific color based on a similar stimulus (e.g. vibration pattern, pressure pattern, surface charge or stress-strain) and the three images can now be subjected to image registration. This means that when the three images are overlapped on top of each other they will give three-fold information on vibrational intensity, which can also be correlated to pressure response in that point (or pixel) and surface charge in that point (or pixel).

FIG. 52 shows the combined VSPC image into a 3D image. Since images where all the layers have been registered and the algorithm assigned the proper color to each layer, it is possible to see a correspondence for every layer which culminates into the identification of a tumor. The comprehensive VSPC 3D map shows the final result very clearly.

Finally, the disclosures in this patent and the explanation of all the combinations of the different maps are not meant to limit the disclosures or the claims to follow. The different maps obtained in this patent do not need to be necessarily executed and created in the same order as explained in EXAMPLE 1, EXAMPLE 2, EXAMPLE 3 and EXAMPLE 4 but rather the final image can also be a combination of, for example, EXAMPLE 1 and EXAMPLE 3 or EXAMPLE 1 and EXAMPLE 4 or EXAMPLE 1, EXAMPLE 2 and EXAMPLE 4 etc. In all cases it will be possible to extract both a 2D and a 3D final image.

Therefore, it is very important to consider that if a laser scanning session of a region of interest is done at a specific depth, not necessarily all maps are needed but just a combination of those would be enough. If, within the same region of interest, there is need to go deeper in the tissue, then the same combination of maps has to, at least, be used before adding another one for consistency reasons. For example if laser scanning a breast surface at 2 centimeter depth, the curing physician decides to use a composition of vibrational map and pressure field that would be acceptable. After the scan is completed he can create a 2D/3D image. If the curing physician would like to do another scan at 2.1 centimeter depth, then the same two maps need to be created at that specific depth within the same region of interest. If the curing physician adds another scan at 2.2 centimeter depth within the same region of interest, he needs to create both a vibrational and pressure field map and can now decide whether to include an additional map such as a stress-strain map he can do so at this step. The final image would be the composition of all the images obtained at various scanning depths.

Methodologies to create a final could be a mathematical interpolation process but also image registration techniques with the various layers. Given the nature and the process it would be possible to predict how the next layer (whether a vibrational, pressure filed, stress-strain or surface charge doesn't matter as long as existing images of that specific map for that specific region of interest already exists) is going to be.

A final example of such techniques would be if the curing physician decides to scan the same patient but at 2.5 centimeters, it is possible to apply a particle filtering technique which, again, is based on the fact of creating a new image based on the previous history of the other images and normalize and update the final image.

### LIST OF EMBODIMENTS AND EXAMPLES

Specific systems and methods of obtaining non-contact images have been described. The detailed description in this specification is illustrative and not restrictive or exhaustive. The detailed description is not intended to limit the disclosure to the precise form disclosed. Other equivalents and modifications besides those already described are possible without departing from the inventive concepts described in this specification, as those skilled in the art will recognize. When the specification or claims recite methods, steps or functions in order, alternative embodiments may perform the tasks in a different order or substantially concurrently. The inventive subject matter is not to be restricted except in the spirit of the disclosure.

When interpreting the disclosure, all terms should be interpreted in the broadest possible manner consistent with the context. Unless otherwise defined, all technical and scientific terms used in this specification have the same meaning as commonly understood by one of ordinary skills in the art to which this invention belongs. This invention is not limited to the particular methodology, systems, protocols, and the like described in this specification and, as such, can vary in practice. The terminology used in this specification is not intended to limit the scope of the invention, which is defined solely by the claims.

What is claimed is:

1. A method for detecting tumor within tissue using vibrational techniques comprising:
a) constructing a vibrational map of the internal surface of the body part by optically detect skin surface waves from an external vibrometer;
b) generating vibrational intensity map from the vibrational surface waves of the body part where the excitation is caused by a pulsed laser;
c) identifying a region of interest from the vibrational intensity map; and
d) detecting suspected tumors within the region of interest.

2. The method of claim 1, wherein constructing a vibrational map comprises assigning vibrational patterns at the detected point and color code the vibrational intensity using an algorithm.

3. The method of claim 1, wherein the vibrational intensities classified as vibrational differences between healthy and cancerous tissue exhibit different vibrational patterns.

4. The method of claim 1, wherein identifying the suspected tumor comprises classifying the vibrational, pressure, stress-strain and surface charge maps obtained at various depths difference between healthy and cancerous tissue.

5. A method for identifying tumor within a tissue of a body part, comprising:
e) generating a 3D digital model of the body part from camera images;
f) obtaining multiple vibrational layers at different depths using a pulsed laser device;
g) obtaining a pressure field image from the vibrational layers;
h) obtaining a surface charge distribution map from the vibrational layers;
i) obtaining a stress-strain map from the vibrational layers;
j) localizing suspected tumors within the body part by associating the vibrational layers with the pressure field map, the surface charge distribution map and the stress-strain stiffness map; and
k) creating a final VSPC 3D image using an image registration process.

6. The method of claim 5, wherein the 3D digital model obtained using cameras is obtained using stereometry techniques from multiple views of the entire body.

7. The method of claim 5, wherein the 3D digital model of the body part comprises internal structures of the body generated from pressure field, stress-strain, surface charge, vibrational intensity, laser ultrasound, ultrasound, MRI, magnetic resonance or x-ray by reconstruction from multiple angles.

8. The method of claim 5, wherein the vibrational map obtained at each layer is aligned with image registration in order to compare various layers and compose a final 2D/3D image of the tumor.

9. A method for identifying tumor within a tissue comprising:
a) generating a database of clinical images of body parts including geometric, vibrational, stress-strain, pressure and surface charge maps of the body parts;
b) detecting geometric and vibrational region of interests of the various body parts;
c) comparing clinical images to other images obtained with vibrational, stress-strain, pressure and surface charge maps;
d) comparing vibrational, stress-strain, pressure and surface charge maps with similar clinical images;
e) detecting tumor progression in the clinical stage;
f) detecting tumor shrinkage in the clinical stage; and
g) detecting no tumor progression in the clinical stage.

10. The method of claim 9, further comprising updating the database with clinical images of body parts.

11. The method of claim 9, wherein a final image can be obtained as a various combination of different maps at the same depth.
